SEQUENCE LISTING (14 sequences)

SEQ ID NO: 1 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 2 - protein, 119 aa, Homo sapiens
  MISC_FEATURE (31)..(35): CDR1
RLQLVQSGAEVRKPGASVKVSCKASGYSFTGHYIHWARQAPGQGLEWMGWINPYSGGTNFPREFQGRVTMTRDTSVNTVYMELTRLTSDDTSVYYCARDHRIVGGLDYWGQGTLVTVSS

SEQ ID NO: 3 - protein, 110 aa, Homo sapiens
  MISC_FEATURE (26)..(35): CDR1
QSVLTQPPSASGTPGQRVTISCSGSSSNLGSNFVYWYQQLPGTAPKLLIYRNHQRPSGVPDRFSGSKSGTSASLAISGLRSEDEADYYCASWDDSLRVVVFGGGTKLTVL

SEQ ID NO: 4 - protein, 119 aa, Homo sapiens
  MISC_FEATURE (31)..(35): CDR1
EVQLVQSGAEVRKPGASVKVSCKASGYSFTGHYIHWVRQAPGQGLEWMGWINPYSGGTNFPREFQGRVTMTRDTSVNTVYMELTRLTSDDTSVYYCARDHRIVGGLDYWGQGTLVTVSS

SEQ ID NO: 5 - protein, 110 aa, Homo sapiens
  MISC_FEATURE (26)..(35): CDR1
SYVLTQPPSASGTPGQRVTISCSGSSSNIGINYVYWYQQLPGTAPKLLIYRNDQRPSGVPDRFSGSKSGTSASLAISGLRSEDEADYYCATWDDSLSGVVFGGGTKVTVL

SEQ ID NO: 6 - protein, 5 aa, Homo sapiens
GHYIH

SEQ ID NO: 7 - protein, 17 aa, Homo sapiens
WINPYSGGTNFPREFQG

SEQ ID NO: 8 - protein, 10 aa, Homo sapiens
DHRIVGGLDY

SEQ ID NO: 9 - protein, 10 aa, Homo sapiens
SSNLGSNFVY

SEQ ID NO: 10 - protein, 7 aa, Homo sapiens
RNHQRPS

SEQ ID NO: 11 - protein, 11 aa, Homo sapiens
ASWDDSLRVVV

SEQ ID NO: 12 - protein, 10 aa, Homo sapiens
SSNIGINYVY

SEQ ID NO: 13 - protein, 7 aa, Homo sapiens
RNDQRPS

SEQ ID NO: 14 - protein, 11 aa, Homo sapiens
ATWDDSLSGVV

(19) United States
(12) Patent Application Publication
(10) Pub. No.: US 2011/0076279 A1
(43) Pub. Date: Mar. 31, 2011

(54) FULLY HUMAN ANTI-VEGF ANTIBODIES AND METHODS OF USING

(75) Inventors: Sumant Ramachandra, Northbrook, IL (US); Walter Robert Bishop, Pompton Plains, NJ (US); Linda Masat, Walnut Creek, CA (US); Chao Bai Huang, San Leandro, CA (US)

(73) Assignees: SCHERING CORPORATION, Kenilworth, NJ (US); XOMA TECHNOLOGY LTD., Hamilton (BM)

(21) Appl. No.: 12/446,438

(22) PCT Filed: Oct. 22, 2007
(86) PCT No.: PCT/US07/82164

§ 371 Date: Dec. 3, 2010

Related U.S. Application Data
(60) Provisional application No. 60/853,260, filed on Oct. 20, 2006.

(51) Int. Cl. A61K 39/395; C07K 16/22; C07H 21/00; C12N 15/63; C12N 5/10; A61P 29/00; A61P 19/02; A61P 17/06; A61P 17/00; A61P 11/00; A61P 11/06; A61P 27/02; A61P 35/00

(52) U.S. Cl. 424/158.1; 530/389.2; 424/178.1; 536/23.53; 435/320.1; 435/336; 530/388.24; 530/387.3

(57) ABSTRACT

Vascular endothelial growth factor (VEGF) overexpression is associated with a variety of conditions involving aberrant angiogenesis. Disclosed herein are fully human antibodies that specifically bind human VEGF and inhibit VEGF binding to VEGF-R1 and VEGF-R2, and therefore inhibit VEGF signaling Based on their ability to inhibit VEGF signaling, the antibodies disclosed herein may be used to treat angiogenesis and conditions associated with angiogenesis both in vivo and in vitro.

### RELATED APPLICATIONS

The present application claims priority to U.S. Provisional Application No. 60/853,260, filed Oct. 20, 2006, which is herein incorporated by reference in its entirety.

### BACKGROUND

The vascular endothelial growth factors (VEGFs) are a major family of angiogenic proteins involved in endothelial cell activation, proliferation, and survival, particularly during retinal proliferative diseases and tumorigenesis. VEGFs belong to the VEGF-PDGF (platelet-derived growth factor) super-gene family, and are small glycoprotein dimers that bind receptors expressed on vascular and lymphatic endothelial cells. There are currently seven known ligands in the VEGF family: VEGF-A (VEGF), VEGF-B, VEGF-C, VEGF-D, VEGF-E (viral-derived), and placental growth factor (PIGF)-1 and -2. These VEGF ligands mediate their effects by binding to one or more of the three known VEGF receptors (VEGF-Rs), each of which possess receptor tyrosine kinase activity. VEGF-R1 (Flt-1) is predominantly expressed on endothelial cells and monocytes, binds VEGF and VEGF-B, and appears to mediate endothelial and monocyte migration. VEGF-R2 (i.e., human KDR or murine Flk-1) is mainly expressed on endothelial cells, is selective for VEGF (and particular fragments of VEGF-C and VEGF-D), and mediates VEGF-induced endothelial cell proliferation, survival, and migration, as well as vascular permeability. VEGF-R3 (Flt-4) is mainly expressed on lymphatic endothelial cells and binds VEGF-C and VEGF-D to promote lymphangiogenesis. VEGF-R1, -R2, and -R3 are each expressed on some tumor cells. Binding of VEGF to the VEGF receptors triggers receptor dimerization, leading to subsequent receptor activation and signal transduction. VEGF binding to VEGF-R2 initiates a signal transduction pathway that is dominant in promoting angiogenesis. This pathway involves receptor activation with subsequent induction of intracellular signaling. Receptor activation in this case entails three basic events: (i) VEGF binding to VEGF-R2, (ii) receptor dimerization, and (iii) receptor autophosphorylation (and hence activation) of the receptor tyrosine kinase. Intracellular messengers such as phospholipase C and phosphatidylinositol-3-kinase bind directly to the autophosphorylated form of VEGF-R2 and become phosphorylated by the receptor tyrosine kinase, which subsequently triggers an intracellular cascade of signaling events leading to nuclear signals that ultimately promote cell proliferation, migration, and survival (anti-apoptosis), and increase vascular permeability.

Aberrant angiogenesis is associated with a variety of disease states, including cancer (Holash 2002). The VEGF pathway is the only signaling pathway that has been verified to play a role in both normal vascular development and the pathologic angiogenesis associated with various diseases (Erikkson 1999; Ferrara 1999; Yancopoulos 2000). VEGF promotes vascular endothelial cell growth and increases vascular permeability (Ferrara 2004).

Previous studies have revealed elevated VEGF expression levels in a majority of tumor types (Berkman 1993; Brown 1993; Brown 1995; Dvorak 1995; Mattern 1996). Studies have also revealed increased VEGF levels in subjects with ocular angiogenic diseases such as wet AMD. Wet AMD accounts for only around 10% of total AMD cases, but causes approximately 90% of blindness arising from AMD. Wet AMD is characterized by choroidal neovascularization (CNV), the development of abnormal blood vessels beneath the retinal pigment epithelium layer of the retina. VEGF-A is believed to play a major role in the formation of these vessels, which leak beneath the macula and cause retinal distortion and vision deterioration.

Several molecules that inhibit the interaction between VEGF and its receptors or that inhibit the tyrosine kinase activity of the VEGF receptors have been developed and are in various stages of clinical development. For example, BAY 43-9006 (Sorafenib, Bayer), SU11248 (Sunitinib), and Vatalanib (Novartis) are VEGF-R2 kinase inhibitors that have been developed as potential cancer therapies. VEGF-TRAP (Regeneron, Sanofi-Aventis) is a hybrid of the extracellular domains of VEGF-R1 and VEGF-R2 with a high binding affinity for VEGF (Holash 2002; Dupont 2005). VEGF-TRAP is currently in clinical trials for the treatment of solid tumors. Antibodies have also been widely developed for use as VEGF pathway inhibitors. For example, IMC-1C11 (Imclone) is a chimeric monoclonal antibody that binds VEGF-R2 (Hunt 2001). Clinical studies are currently being conducted to determine the efficacy of IMC-1C11 in the treatment of metastatic colorectal carcinoma. CDP-791 (Imclone) is a PEGylated humanized Fab that binds VEGF-R2. IMC-1121B (Imclone) is a fully human monoclonal antibody that binds VEGF-R2 with high affinity and blocks the interaction between VEGF and VEGF-R2 (Miao, H. Q., et al. 2005. Biochem Biophys Res Commun 345:438-445). IMC-18F1 (Imclone) is a fully human monoclonal antibody that binds VEGF-R1 with high affinity and blocks the interaction between VEGF and VEGF-R1. 2C3 (Peregrine) is a mouse antibody that binds VEGF and blocks binding between VEGF and VEGF-R2, but not VEGF-R1. Ranibizumab (Lucentis®, Genentech) is a humanized Fab derived from Bevacizumab that was recently approved for the treatment of wet acute macular degeneration (AMD) (Michels 2004; Rosenfeld 2005).

The most commonly used biological therapeutic targeting VEGF is the humanized IgG1 monoclonal antibody Bevacizumab (a.k.a., Avastin®, Genentech; also referred to herein as BM-1). Bevacizumab was developed by humanizing the mouse monoclonal antibody A.4.6.1, and it contains approximately 93% human sequence and 7% murine sequence (Presta 1997; Ferrara 2004). Bevacizumab binds VEGF with an affinity of approximately 500 pM, and inhibits binding of VEGF to VEGF-R1 and VEGF-R2. The terminal half-life of Bevacizumab in humans is 17-21 days (Ferrara 2004). Bevacizumab has been approved for the treatment of metastatic colorectal cancer (Presta 1997; Rosenfeld 2005). Studies have also suggested that Bevacizumab may be useful in the treatment of neovascular AMD (Michels 2005).

Despite the variety of molecules that have been developed for inhibition of the VEGF pathway, there is a need for additional molecules with improved binding characteristics and therapeutic profiles. The present invention provides fully human antibodies that bind VEGF and inhibit the VEGF pathway. Since these antibodies are fully human, they can be administered as therapeutic agents without the adverse immunogenicity associated with previously developed non-human antibodies, chimeric antibodies, or humanized antibodies.

### SUMMARY

In certain embodiments, an antibody is provided that comprises the heavy chain sequence set forth in SEQ ID NO:2 and the light chain sequence set forth in SEQ ID NO:3. In certain other embodiments, an antibody is provided that comprises the heavy chain sequence set forth in SEQ ID NO:4 and the light chain sequence set forth in SEQ ID NO:5. In certain of the above embodiments, the antibody comprises an IgG2 constant region. In certain embodiments, the antibody binds hVEGF₁₆₅ with a K_{D} of ≦2.0 nM. In certain embodiments, the antibody binds to an epitope on hVEGF₁₆₅ that overlaps with the epitope bound by Bevacizumab. In certain embodiments, the antibody blocks binding of hVEGF₁₆₅ to VEGF-R1 and VEGF-R2.

In certain embodiments, methods are provided for inhibiting angiogenesis in a subject in need thereof by administering to said subject a therapeutically effective amount of an antibody that comprises the heavy chain sequence set forth in SEQ ID NO:2 and the light chain sequence set forth in SEQ ID NO:3. In certain other embodiments, methods are provided for inhibiting angiogenesis in a subject in need thereof by administering to said subject a therapeutically effective amount of an antibody that comprises the heavy chain sequence set forth in SEQ ID NO:4 and the light chain sequence set forth in SEQ ID NO:5.

In certain embodiments, methods are provided for treating a disease associated with aberrant angiogenesis in a subject in need thereof comprising administering to said subject a therapeutically effective amount of an antibody that comprises the heavy chain sequence set forth in SEQ ID NO:2 and the light chain sequence set forth in SEQ ID NO:3. In certain other embodiments, methods are provided for treating a disease associated with aberrant angiogenesis in a subject in need thereof by administering to said subject a therapeutically effective amount of an antibody that comprises the heavy chain sequence set forth in SEQ ID NO:4 and the light chain sequence set forth in SEQ ID NO:5.

In certain embodiments, methods are provided for treating an inflammatory disease associated with VEGF signaling in a subject in need thereof comprising administering to said subject a therapeutically effective amount of an antibody that comprises the heavy chain sequence set forth in SEQ ID NO:2 and the light chain sequence set forth in SEQ ID NO:3. In certain other embodiments, methods are provided for treating an inflammatory disease associated with VEGF signaling in a subject in need thereof by administering to said subject a therapeutically effective amount of an antibody that comprises the heavy chain sequence set forth in SEQ ID NO:4 and the light chain sequence set forth in SEQ ID NO:5.

In certain embodiments, methods are provided for treating wet acute macular degeneration or diabetic retinopathy in a subject in need thereof comprising administering to said subject a therapeutically effective amount of an antibody that comprises the heavy chain sequence set forth in SEQ ID NO:2 and the light chain sequence set forth in SEQ ID NO:3. In certain other embodiments, methods are provided for treating wet acute macular degeneration or diabetic retinopathy in a subject in need thereof by administering to said subject a therapeutically effective amount of an antibody that comprises the heavy chain sequence set forth in SEQ ID NO:4 and the light chain sequence set forth in SEQ ID NO:5.

In certain embodiments, methods are provided for treating a cancer associated with increased VEGF signaling in a subject in need thereof comprising administering to said subject a therapeutically effective amount of an antibody that comprises the heavy chain sequence set forth in SEQ ID NO:2 and the light chain sequence set forth in SEQ ID NO:3. In certain other embodiments, methods are provided for treating a cancer associated with increased VEGF signaling in a subject in need thereof by administering to said subject a therapeutically effective amount of an antibody that comprises the heavy chain sequence set forth in SEQ ID NO:4 and the light chain sequence set forth in SEQ ID NO:5.

In certain embodiments, a kit is provided comprising an antibody that comprises the heavy chain sequence set forth in SEQ ID NO:2 and the light chain sequence set forth in SEQ ID NO:3. In certain other embodiments, a kit is provided comprising an antibody that comprises the heavy chain sequence set forth in SEQ ID NO:4 and the light chain sequence set forth in SEQ ID NO:5.

In certain embodiments, polynucleotides are provided that encode the amino acid sequence set forth in SEQ ID NO:2, SEQ ID NO:3, SEQ ID NO:4, and/or SEQ ID NO:5. In certain other embodiments, vectors are provided that comprise these polynucleotides, and in certain other embodiments, host cells are provided that comprises these vectors.

### BRIEF DESCRIPTION OF THE DRAWINGS

FIG. 1: Heavy chain variable regions (including HCDRs) and light chain variable regions (including LCDRs) of XPA.10.064 and XPA.10.072

FIG. 2: Biacore analysis of XPA.10.064 IgG2 binding to human hVEGF₁₆₅.

FIG. 3: Biacore analysis of XPA.10.072 IgG2 binding to human hVEGF₁₆₅.

FIG. 4: Biacore analysis of Bevacizumab binding to human hVEGF₁₆₅.

FIG. 5: Inhibition of hVEGF₁₆₅ binding to VEGF-R1 by Bevacizumab (BM1), XPA.10.064, and XPA.10.072.

FIG. 6: Inhibition of hVEGF₁₆₅ binding to VEGF-R2 by Bevacizumab (BM1), XPA.10.064, and XPA.10.072.

FIG. 7: Analysis of hVEGF₁₆₅ epitopes bound by Bevacizumab (BM1), XPA.10.064 (064), and XPA.10.072 (072).

FIG. 8: Co-localization of XPA.10.064 and G153-694Panel A is shows the staining with XPA.10.064. Panel B shows staining with G153-694. Panel C shows staining with a nuclear dye. Panel D shows the merged image, where greater intensity (white) reflects colocalization.

FIG. 9: Co-localization of XPA.10.072 and G153-694. Panel A is shows the staining with XPA.10.072. Panel B shows staining with G153-694. Panel C shows staining with a nuclear dye. Panel D shows the merged image, where greater intensity (white) reflects colocalization.

FIG. 10: Inhibition of HUVEC proliferation by (A) XPA.10.064 IgG2 and (B) XPA.10.072 IgG2.

FIG. 11: Treatment of HUVECs with a dose titration of hVEGF₁₆₅ leads to an increase in phosphorylation of VEGF-R2.

FIG. 12: Treatment of HUVECs with a dose titration of hVEGF₁₆₅ plus Bevacizumab resulted in a decrease in VEGF-R2 phosphorylation.

FIG. 13: XPA.10.064 (064) and XPA.10.072 (072) IgG2s inhibit hVEGF₁₆₅-induced phosphorylation of VEGF-R2.

FIG. 14: Visual Scoring for Matrigel Plugs. The visual scoring was developed as hemoglobin assay for PH07-VAL-021 did not capture what was seen in the plugs.

FIG. 15: Matrigel plug assay showing the level of angiogenesis inhibition in the presence of Bevacizumab (BM-1), XPA.10.064 (064), and XPA.10.072 (072) at various dosages.

FIG. 16: Inhibition of angiogenesis as determined by Matrigel plug assay. Numbers are the mean of results from two blinded scorers. (A) no cells; (B) DU145 plus αKLH; (C) Bevacizumab (0.1 mg/kg); (D) Bevacizumab (1 mg/kg); (E) Bevacizumab (5 mg/kg); (F) XPA.10.064 (0.1 mg/kg); (G) XPA.10.064 (1 mg/kg); (H) XPA.10.064 (5 mg/kg); (I) XPA.10.072 (0.1 mg/kg); (J) XPA.10.072 (1 mg/kg); (K) XPA.10.072 (5 mg/kg).

FIG. 17: Inhibition of A673 tumor growth in vivo by XPA.10.064 and XPA.072. (1) vehicle only; (2) 0.5 mg/kg XPA.10.064 IgG2; (3) 5 mg/kg XPA.10.064 IgG2; (4) 0.5 mg/kg XPA.10.072 IgG2, (5) 5 mg/kg XPA.10.072 IgG2; (6) 5 mg/kg CHO.KLH IgG2; (7) 0.5 mg/kg Bevacizumab; (8) 5 mg/kg Bevacizumab (Group 8).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

The following abbreviations are used herein: ADCC, antibody-dependent cellular cytotoxicity; AMD, age-related macular degeneration; CDC, complement-dependent cytotoxicity; CNV, choroidal neovascularization; COPD, chronic obstructive pulmonary disease; HUVEC, human umbilical vein endothelial cell; hVEGF, human VEGF; mVEGF, murine VEGF; PD, pharmacodynamics; PK, pharmacokinetics; RA, rheumatoid arthritis; RIA, radioimmunoprecipitation; VEGF, vascular endothelial growth factor; VEGF-R, vascular endothelial growth factor receptor; VHL, von Hippel/Lindau; X-reactivity, cross-reactivity.

### Definitions

The term “antibody” as used herein refers to any immunoglobulin, including a monoclonal antibody, polyclonal antibody, or multispecific or bispecific antibody, that binds to a specific antigen. A complete antibody comprises two heavy chains and two light chains. Each heavy chain consists of a variable region and a first, second, and third constant region, while each light chain consists of a variable region and a constant region. The antibody has a “Y” shape, with the stem of the Y consisting of the second and third constant regions of two heavy chains bound together via disulfide bonding. Each arm of the Y consists of the variable region and first constant region of a single heavy chain bound to the variable and constant regions of a single light chain. The variable regions of the light and heavy chains are responsible for antigen binding. The variable region in both chains generally contains three highly variable loops called the complementarity determining regions (CDRs) (light (L) chain CDRs including LCDR1, LCDR2, and LCDR3, heavy (H) chain CDRs including HCDR1, HCDR2, HCDR3). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The major classes of antibodies are IgA, IgD, IgE, IgG, and IgM, with several of these classes divided into subclasses such as IgG1, IgG2, IgG3, IgG4, IgA1, or IgA2.

The term “antigen-binding fragment” as used herein further refers to an immunoglobulin fragment or an antibody fragment thereof (i.e., at least one immunologically active portion of an immunoglobulin molecule), such as a Fab, Fab′, F(ab′)₂, Fv fragment, a single-chain antibody molecule, a multispecific antibody, formed from any fragment of an immunoglobulin molecule comprising one or more CDRs. In addition, an antigen-binding fragment as used herein may comprise one or more CDRs from a particular human immunoglobulin grafted to a framework region from one or more different human immunoglobulins. The antigen-binding fragment is capable of binding to the target to which the parent immunoglobulin or antibody binds.

“Fab” with regards to an antibody refers to that portion of the antibody consisting of a single light chain (both variable and constant regions) bound to the variable region and first constant region of a single heavy chain by a disulfide bond.

“Fab” refers to a Fab fragment that includes a portion of the hinge region.

“F(ab′)₂ refers to a dimer of Fab.

“Fc” with regards to an antibody refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

“Fv” with regards to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Houston 1988).

“Single-chain Fv-Fc antibody” or “scFv-Fc” refers to an engineered antibody consisting of a scFv connected to the Fc region of an antibody.

The term “epitope” as used herein refers to the group of atoms or amino acids on an antigen molecule to which an antibody binds. As used herein, two antibodies bind the same epitope within an antigen if they exhibit competitive binding for the antigen. For example, if an antibody as disclosed herein competes with Bevacizumab for VEGF binding, the antibody is considered to bind the same epitope as Bevacizumab.

“VEGF” or “VEGF ligand” as used herein refers to one of the seven currently known VEGF ligands: VEGF-A, VEGF-B, VEGF-C, VEGF-D, VEGF-E (viral-derived), or placental growth factor (PIGF)-1 or -2. With regard to VEGF-A, there are currently four known splicing isoforms, with each demonstrating unique biological functions. The 165 amino acid isoform (VEGF₁₆₅, SEQ ID NO:1) exists in both heparin-bound and soluble forms. The 121 amino acid isoform (VEGF₁₂₁), which is missing a fragment corresponding to the region between residues 115 and 159 of VEGF₁₆₅, exists in soluble form only. The longer 189 and 206 amino acid isoforms (VEGF₁₈₉ and VEGF₂₀₆, respectively) retain the ability to bind heparin. The antibodies provided herein exhibit high binding affinity for human VEGF₁₆₅, but in certain embodiments may cross-react or exhibit low-level binding affinity to a non-human VEGF protein or to other VEGF isoforms.

“Treating” or “treatment” of a condition as used herein may refer to preventing or alleviating the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. With regard to tumors, “treating” or “treatment” may refer to eradicating all or part of a tumor, inhibiting or slowing tumor growth and metastasis, preventing or delaying the development of a tumor, or some combination thereof.

The term “specifically binds” as used herein refers to a non-random binding reaction between two molecules, such as for example between an antibody and a ligand to which the antibody is raised. As used herein, an antibody that specifically binds a first ligand may exhibit cross-reactivity or low level binding affinity with a second ligand. In certain embodiments, an antibody that specifically binds a ligand binds the ligand with a binding affinity (K_{D}) of ≦10⁻⁷ M (e.g., 5×10⁻⁸ M, 10⁻⁸ M, 5×10⁻⁹ M, 10⁻⁹ M, 10⁻¹⁰ M). K_{D}, which as used herein refers to the ratio of the dissociation rate to the association rate (k_{off}/kₒₙ), may be determined using methods known in the art. In other embodiments, an antibody specifically binds to a first ligand with a binding affinity of no less than 10 folder (e.g., ≧15 folder, ≧20 folder, ≧50 folder, ≧10² folder, ≧10³ folder, or ≧10⁴ folder) higher or lower than the binding affinity with which the antibody binds to a second ligand. In other embodiments, an antibody that specifically binds a ligand binds the ligand with a binding affinity (K_{D}) of ≦10⁻⁷ M, but exhibit little binding affinity with a second ligand.

The term “isolated” as used herein means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. “Isolated” as used herein does not exclude artificial or synthetic mixtures with other compounds or materials, or the presence of impurities that do not interfere with activity.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The phrase “host cell” as used herein refers to a cell into which a vector has been introduced. A host cell may be selected from a variety of cell types, including for example bacterial cells such as E. coli or B. subtilis cells, fungal cells such as yeast cells or Aspergillus cells, insect cells such as Drosophila S2 or Spodoptera Sf9 cells, or animal cells such as fibroblasts, CHO cells, COS cells, NSO cells, HeLa cells, BHK cells, HEK 293 cells, or human cells.

A “disease associated with aberrant angiogenesis” as used herein refers to any condition that is caused by, exacerbated by, or otherwise linked to increased angiogenesis, specifically increased angiogenesis associated with the VEGF signaling pathway. Such conditions include cancer types that are dependent on neo-angiogenesis for growth, diseases of the eye such as for example wet AMD, and inflammatory conditions such as for example rheumatoid arthritis, psoriasis, scleroderma, chronic obstructive pulmonary disease, or asthma.

The ability to “block binding” or “compete binding” as used herein refers to the ability of an antibody to inhibit the binding interaction between two molecules by 50% or greater. In certain embodiments, this inhibition may be greater than 60%, in certain embodiments greater than 70%, in certain embodiments greater than 80%, and in certain embodiments greater than 90%. In certain embodiments, the binding interaction being inhibited may be that of Bevacizumab to hVEGF₁₆₅. In certain other embodiments, the interaction being inhibited may be that of hVEGF₁₆₅ to VEGF-R1 and/or VEGF-R2.

The term “therapeutically effective amount” as used herein refers to the amount or concentration of a drug effective to treat a disease or condition. For example, with regard to the use of the antibodies disclosed herein to treat cancer, a therapeutically effective amount of an antibody is the dosage or concentration of the antibody capable of eradicating all or part of a tumor, inhibiting or slowing tumor growth and metastasis, preventing or delaying the development of a tumor, or some combination thereof. For another example, the dosage for treatment ranges from about 0.01 mg/kg to about 100 mg/kg, e.g., between about 0.01 mg/kg and about 50 mg/kg (e.g., about 0.1 mg/kg, 1 mg/kg, or 10 mg/kg). In certain embodiments, the initial treatment dosage does not exceed about 100 mg/kg or 50 mg/kg, and subsequent treatment dosage is at least 0.01 mg/kg. A given dosage can be administered daily, bi-weekly, weekly, or monthly.

### Fully Human VEGF Antibodies

Fully human antibodies have several potential advantages over murine, chimeric, or humanized antibodies in terms of both safety and efficacy. First, their lack of non-human residues makes fully human antibodies less likely to generate a host immune response following administration. Second, fully human antibodies generally exhibit lower clearance rates than other antibody types. This decreased clearance rate allows for the use of lower dosage amounts and frequencies.

Provided herein are two fully human antibodies, XPA.10.064 and XPA.072, that specifically bind hVEGF₁₆₅. XPA.10.064 and XPA.10.072 were identified by panning a phage display scFv library with hVEGF₁₆₅. The heavy and light chain variable region sequences of XPA.10.072 are set forth in SEQ ID NOs: 2 and 3, respectively, and the heavy and light chain variable region sequences of XPA.10.064 are set forth in SEQ ID NOs:4 and 5, respectively. The XPA.10.072 heavy chain variable region as set forth in SEQ ID NO:2 contains CDRs at residues 31-35 (CDRH1, SEQ ID NO:6), 50-66 (CDRH2, SEQ ID NO:7), and 99-108 (CDRH3, SEQ ID NO:8). The XPA.10.072 light chain variable region as set forth in SEQ ID NO:3 contains CDRs at residues at residues 26-35(072CDRL1, SEQ ID NO:9), 51-57 (072CDRL2, SEQ ID NO:10), and 90-100 (072CDRL3, SEQ ID NO:11). The XPA.10.064 heavy chain variable region as set forth in SEQ ID NO:4 contains CDRs at residues 31-35 (CNRH1, SEQ ID NO:6), 50-66 (CDRH2, SEQ ID NO:7), and 99-108 (CDRH3, SEQ ID NO:8). The XPA.10.064 light chain variable region as set forth in SEQ ID NO:5 contains CDRs at residues at residues 26-35 (064CDRL1, SEQ ID NO:12), 51-57 (064CDRL2, SEQ ID NO:13), and 90-100 (064CDRL3, SEQ ID NO:14).

Based on the ability of XPA.10.064 and XPA.10.072 scFvs to bind hVEGF₁₆₅ with high affinity in an ELISA and to inhibit binding of hVEGF₁₆₅ to VEGF-R1 and VEGF-R2, the antibodies were selected for conversion to scFv-Fc and IgG2 for additional functional studies. XPA.10.064 and XPA.10.072 IgG2s exhibited similar high binding affinity for hVEGF₁₆₅ as determined by Biacore analysis, while displaying only weak binding with mVEGF₁₆₅. Both antibodies also bind hVEGF₁₂₁. The binding affinities of XPA.10.064 and XPA.10.072 IgG2s for hVEGF₁₆₅ were 1.5 nM and 1.7 nM, respectively, and both antibodies exhibited off-rates that were approximately twice as fast as Bevacizumab. Biacore analysis also revealed that XPA.10.064 and XPA.10.072 IgG2s block binding of hVEGF₁₆₅ to VEGF-R1 and VEGF-R2 to a higher degree than Bevacizumab. The ability of XPA.10.064 and XPA.10.072 to inhibit VEGF signaling was confirmed by ELISA experiments showing that both antibodies inhibit h₁₆₅VEGF-induced VEGF-R2 phosphorylation.

Epitope analysis revealed that XPA.10.064 and XPA.10.072 bind linear epitopes on hVEGF₁₆₅, and that these epitopes overlap to at least some degree with the epitopes bound by Bevacizumab. Immunohistochemical analysis revealed that, unlike Bevacizumab, XPA.10.064 and XPA.10.072 both exhibit broad range tissue cross-reactivity. Both antibodies inhibit HUVEC proliferation, and both inhibit angiogenesis and tumor growth in vivo. A summary of the characteristics of XPA.10.064 and XPA.10.072 is set forth in Table 1.

Therefore, provided herein in certain embodiments are antibodies comprising the heavy and light chains of XPA.10.064 as set forth in SEQ ID NOs:4 and 5, respectively. In other embodiments, antibodies are provided that comprise the heavy and light chains of XPA.10.072 as set forth in SEQ ID NO:2 and 3, respectively. Based on their high binding affinity for hVEGF₁₆₅ and hVEGF₁₂₁ and their ability to block VEGF binding to VEGF-R1 and VEGF-R2 and VEGF-induced receptor phosphorylation, the antibodies provided herein may be used to inhibit VEGF signaling and block the VEGF pathway. On this basis, the antibodies may be used to treat various conditions associated with VEGF expression.

The antibodies provided herein have been found to inhibit HUVEC proliferation and to inhibit angiogenesis in vitro. Therefore, the antibodies may be used to treat various conditions associated with increased angiogenesis. For example, the antibodies may be used to treat cancer by inhibiting the proliferation of blood vessels from a tumor site and thus inhibiting tumor growth. Likewise, the antibodies may used to treat cancer by destroying blood vessels at a tumor site, resulting in tumor necrosis. The efficacy of the antibodies disclosed herein for the treatment of cancer has been confirmed in vivo.

In certain embodiments, the antibodies provided herein may inhibit angiogenesis and/or tumor growth at a level similar to or to a greater extent than Bevacizumab. In an in vivo A673 tumor growth inhibition experiment, XPA.10.064 and XPA.10.072 both inhibited A673 tumor growth at a level similar to Bevacizumab at all dosages tested. In certain embodiments, the antibodies disclosed herein may inhibit angiogenesis and/or tumor growth at a level equal to or greater than that of Bevacizumab when administered at a lower dosage or concentration. The antibodies provided herein may exhibit similar or improved pharmacokinetic (PK) properties as compared to Bevacizumab. For example, the antibodies may exhibit increased circulating half-life or decreased immunogenicity as compared to Bevacizumab. In certain embodiments wherein the antibodies exhibit similar or improved pharmacokinetic properties versus Bevacizumab, the antibodies may be administered over a longer interval than Bevacizumab without exhibiting negative effects associated with increased intervals of Bevacizumab administration.

The antibodies disclosed herein may be used in the treatment of any condition associated with aberrant angiogenesis controlled at least in part by the VEGF pathway. These conditions, which are generally associated with increased VEGF expression levels, include ocular diseases associated with increased angiogenesis, such as wet AMD or proliferative retinopathies such as diabetic retinopathy, diabetic kidney disease and other diabetic vascular proliferative diseases, cystic fibrosis, and various tumor types (Amoroso 1997; McColley 2000; Khamaisi 2003).

Tumor types that may be treated using the antibodies disclosed herein include but are not limited to carcinoma, lymphoma, blastoma, sarcoma, leukemia, or lymphoid malignancies. More specifically, tumor types that may be treated using the antibodies disclosed herein include but are not limited to squamous cell cancer such as for example epithelial squamous cell cancer, lung cancer including small cell lung cancer and non-small cell lung cancer, cancer of the peritoneum, hepatocellular cancer, gastric or stomach cancer such as for example gastrointestinal cancer, pancreatic cancer, glioblastoma, cervical cancer, ovarian cancer, liver cancer, bladder cancer, hepatoma, endometrial or uterine carcinoma, salivary gland carcinoma, kidney or renal cancer, prostate cancer, breast cancer, colon cancer, rectal cancer, colorectal cancer, vulval cancer, thyroid cancer, hepatic carcinoma, anal carcinoma, penile carcinoma, head and neck cancer, and pediatric cancers such as pediatric sarcomas. In addition, tumors can be malignant (e.g., cancers) or benign (e.g., hyperplasia, cyst, pseudocyst, hamartoma, and benign neoplasm).

Tumor types that may be treated using the antibodies disclosed herein also include cancers associated with a particular biomarker. For example, a biomarker includes, but is not limited to, mutations in the von Hippel-Lindau (VHL) tumor suppressor gene and/or overexpression of Hypoxia-inducible factor-1α (HIF-1α). In certain embodiments, the antibodies can be used to treat cancers displaying mutations in the VHL tumor suppressor gene. Mutations in the VHL gene result in the constitutive stabilization of hypoxia-inducible transcription factors 1α and 2α, which bind to enhancer elements in the VEGF gene and stimulate angiogenesis (Harris 2000). VHL mutant tumor types that may be treated using the antibodies disclosed herein include, for example, central nervous system hemangioblastomas, retinal hemangioblastomas, endolymphatic sac tumors, clear cell renal cell cancers and/or renal cysts, pheochromocytomas, pancreatic cysts, neuroendocrine tumors, and epididymal and broad ligament cystadenomas. Subjects are first selected or screened for the presence of VHL gene mutations through known methods such as the molecular detection using a mutation specific nested reverse transcription polymerase chain reaction or a nested single strand conformational polymorphism analysis (Ashida et al., J. Urol. 169:20898-93 (2003)). The identified subjects are then subject to the treatment with the antibodies according to the present invention. In other embodiments, the antibodies can be used to treat cancers displaying overexpression of HIF-1α in a subject. The HIF-1α overexpression can be examined through biopsies of a tissue (e.g., brain, breast, cervical, esophageal, oropharyngeal, ovarian, and prostate tissues). The identified subjects are selected and subject to the treatment with the antibodies or the antibodies in combination with HIF-1α inhibitors such as 2-methoxyestradiol, 4-O-methylsarcerneol, manassantin A, manassantin B1, NSC-134754, NSC-643735, topotecan, SCH66336, PX-478, R115777, Cetuximab, 103D5R, and NSAID (See Kimbro & Simons, Endocrine-Related Cancer, 13:739-749 (2006)).

Other conditions that may be treated by the antibodies described herein include inflammatory conditions such as rheumatoid arthritis, psoriasis, scleroderma, chronic obstructive pulmonary disease, and asthma. In certain embodiments, the antibodies provided herein may be used to treat a condition that has become resistant to treatment with Bevacizumab.

The antibodies provided herein may be utilized in various non-therapeutic uses. In certain embodiments, the antibodies may be used as affinity purification agents to purify hVEGF₁₆₅, other VEGF isoforms, or fragments thereof. In these embodiments, the antibodies may be immobilized on a solid phase such as a resin or filter paper using methods known in the art. The antibodies may also be used to precipitate hVEGF₁₆₅, other VEGF isoforms, or fragments thereof from solution. In other non-therapeutic embodiments, the antibodies may be used in various in vitro or in vivo diagnostic or detection applications. In certain of these embodiments, the antibodies may be conjugated to a detectable label. In other embodiments, the antibodies may not be conjugated to a detectable label, but may be detected using a labeled antibody that binds to the antibody. In certain embodiments, the antibodies disclosed herein may be used to detect hVEGF₁₆₅ expression. In certain of these embodiments, the antibodies may be used to diagnose a condition associated with increased hVEGF₁₆₅ expression. For example, the antibody may be contacted with a biological sample from a subject in order to diagnose a condition associated with increased hVEGF₁₆₅ in the subject. Likewise, the antibody may be administered to the subject directly, and binding to hVEGF₁₆₅ is detected using methods known in the art.

Mutant epitope binding studies show that the antibodies disclosed herein bind linear epitopes on VEGF that overlap at least partially with the epitope recognized by Bevacizumab. Therefore, in certain embodiments, the antibodies disclosed herein may bind to an epitope consisting of or comprising residues 79-94 of hVEGF₁₆₅ (SEQ ID NO:1). Likewise, the antibodies may bind an epitope that completely or partially overlaps with the sequence corresponding to residues 79-94 of SEQ ID NO:1. Based on the overlap in epitopes, in certain embodiments the antibodies disclosed herein may competitively inhibit Bevacizumab binding to hVEGF₁₆₅.

The antibodies provided herein preferably may have a terminal half-life (T_{1/2}) in humans that is similar to or greater than that of Bevacizumab, which has a half-life of 17-21 days. Terminal half-life, which refers to the time that it takes the plasma concentration of an administered antibody to decrease by one half, may be calculated using methods known in the art. In certain embodiments, the terminal half-life of the antibodies disclosed herein may be at least 17 days. In certain other embodiments, it may be 17-21 days, and in certain of these embodiments it may be greater than 21 days.

The antigen-binding fragments disclosed herein may comprise a fragment or fragments of a full-length antibody, such as for example a Fab, Fab′, F(ab′)₂, Fv, or scFv fragment. These fragments can be produced from full-length antibodies using methods well known in the art, such as for example proteolytic cleavage with enzymes such as papain to produce Fab fragments or pepsin to produce F(ab′)₂ fragments. In certain embodiments, the antibodies disclosed herein may comprise one or more CDRs from SEQ ID NOs:2-5 grafted to one or more human framework regions.

Those of skill in the art will recognize that a variety of conjugates may be coupled to or associated with or used in combination with the antibodies provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies by covalent binding, affinity binding, intercalation, coordinate binding, complexation, association, blending, or addition, among other methods. In certain embodiments, the antibodies of the present invention may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate. In certain embodiments, the antibodies may be linked to a conjugate indirectly, or through another conjugate. For example, the antibody may be conjugated to biotin, then indirectly conjugated to a second conjugate that is conjugated to avidin.

In certain embodiments, conjugates linked to or used in combination with the antibodies disclosed herein may comprise one or more agents meant to alter one or more pharmacokinetic (PK) properties of the antibody, such as for example polyethylene glycol (PEG) to increase the half-life or decrease the immunogenicity of the antibody (see, e.g., Katre 1990).

In certain embodiments, conjugates may comprise one or more cytokines, which as used herein refers to any protein that acts on a cell as an intercellular mediator. Example of cytokines include but are not limited to lymphokines, monokines, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, fibroblast growth factor, prolactin, placental lactogen, tumor necrosis factor α and β, mullerian-inhibiting substance, mouse gonadotropin-associated peptide, inhibin, activin, integrin, thrombopoietin (TPO), nerve growth factors such as NGF-β, platelet growth factor, transforming growth factors such as TGF-α and TGF-β, insulin-like growth factor I and II, erythropoietin (EPO), osteoinductive factors, interferons such as interferon-α, -β, and -γ, colony stimulating factors such as macrophage-CSF, granulocyte macrophage CSF, and granulocyte-CSF, interleukins such IL-1, IL-1α, IL-2, IL-3, IL-4, IL-5, IL-6, IL-7, IL-8, IL-9, IL-10, IL-11, and IL-12, tumor necrosis factors such as TNF-α and TNF-β, and other polypeptide factors. The antibodies and antigen-binding fragments thereof of the invention may be provided and/or administered in combination with any of the foregoing cytokines.

In certain embodiments, conjugates linked to or used in combination with the antibodies disclosed herein may comprise one or more chemotherapeutic agents, which as used herein refers to any agent useful in the treatment of cancer. Examples of chemotherapeutic agents include, but are not limited to, temozolomide, an antibody that binds specifically to IGF1 receptor, lonafarnib, calcitriol, temsirolimus, irinotecan, camptothecin, doxurubicin, adriamycin, toxins such as for example ricin, diptheria toxin, or another toxin of bacterial, fungal, plant or animal origin, coagulants, alkylating agents such as for example thiotepa and cyclosphosphamide (CYTOXAN™), alkyl sulfonates such as for example busulfan, improsulfan and piposulfan, aziridines such as for example benzodopa, carboquone, meturedopa, and uredopa, ethylenimines and methylamelamines such as altretamine, triethylenemelamine, trietylenephosphoramide, triethylenethiophosphaoramide and trimethylolomelamine, nitrogen mustards such as for example chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard, nitrosureas such as carmustine, chlorozotocin, fotemustine, lomustine, nimustine, ranimustine, antibiotics such as for example aclacinomysins, actinomycin, authramycin, azaserine, bleomycins, cactinomycin, calicheamicin, carabicin, camomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as for example methotrexate and 5-fluorouracil (5-FU), folic acid analogues such as for example denopterin, methotrexate, pteropterin, trimetrexate, purine analogs such as for example fludarabine, 6-mercaptopurine, thiamiprine, thioguanine, pyrimidine analogs such as for example ancitabine, azacitidine, 6-azauridine, carmofur, cytarabine, dideoxyuridine, doxifluridine, enocitabine, floxuridine, 5-FU, androgens such as for example calusterone, dromostanolone propionate, epitiostanol, mepitiostane, testolactone, anti-adrenals such as for example aminoglutethimide, mitotane, trilostane, folic acid replenishers such as for example frolinic acid, aceglatone, aldophosphamide glycoside, aminolevulinic acid, amsacrine, bestrabucil, bisantrene, edatraxate, defofamine, demecolcine, diaziquone, elformithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, PSK™, razoxane, sizofiran, spirogermanium, tenuazonic acid, triaziquone, 2,2′,2″-trichlorotriethylamine, urethane, vindesine, dacarbazine, mannomustine, mitobronitol, mitolactol, pipobroman, gacytosine, arabinoside (“Ara-C”), cyclophosphamide, thiotepa, taxanes such as for example paclitaxel (TAXOL™, Bristol-Myers Squibb Oncology, Princeton, N.J.) and docetaxel (TAXOTERE™, Rhone-Poulenc Rorer, Antony, France), ABRAXANE® (paclitaxel albumin-bound particles for injectable suspension with particle size of approximately 130 nm), chlorambucil, gemcitabine, 6-thioguanine, mercaptopurine, methotrexate, platinum analogs such as for example cisplatin and carboplatin, satraplatin, oxaliplatin, vinblastine, platinum, etoposide (VP-16), ifosfamide, mitomycin C, mitoxantrone, vincristine, vinorelbine, navelbine, novantrone, teniposide, daunomycin, aminopterin, xeloda, ibandronate, CPT-11, topoisomerase inhibitor RFS 2000, difluoromethylornithine (DMFO), retinoic acid, esperamicins, capecitabine, gemcitabine, and pharmaceutically acceptable salts, acids, and/or derivatives of any of the above. Chemotherapeutic agents may also include anti-hormonal agents that act to regulate or inhibit hormone action on tumors such as for example anti-estrogens including tamoxifen, raloxifene, aromatase inhibiting 4(5)-imidazoles, 4-hydroxytamoxifen, trioxifene, keoxifene, LY117018, onapristone, and toremifene (Fareston), and anti-androgens including flutamide, nilutamide, bicalutamide, leuprolide, and goserelin. The antibodies and antigen-binding fragments thereof of the invention may be provided and/or administered in combination with any of the foregoing chemotherapeutic agents.

In certain embodiments, conjugates linked to the antibodies disclosed herein may comprise one or more detectable labels. Such labels include, but are not limited to, radioactive isotopes such as ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels, fluorescent labels such as for example fluorescein, rhodamine, dansyl, phycoerythrin, or Texas Red, and enzyme-substrate labels such as for example horseradish peroxidase, alkaline phosphatase, or β-D-galactosidase.

For treatment of diseases associated with high VEGF expression levels and/or increased angiogenesis, the antibodies of the present invention may be prepared at an effective dose as a formulation within pharmaceutically acceptable media. This formulation may include physiologically tolerable liquids, gels, solid carriers, diluents, adjuvants, or excipients, or some combination thereof. Effective dosage will depend in part on the weight, age, and state of health of the subject, as well as the administration route and extent of tumor development. The pharmaceutical formulation containing the antibody may be administered alone or in combination with other known therapeutic agents. For example, the antibodies may be administered in combination with any therapeutic agent known to inhibit the VEGF pathway, including other antibodies or antibody-based therapeutics. Likewise, the antibodies may be administered in combination with therapeutic compounds that do not inhibit the VEGF pathway, but instead target another pathway or indication associated with a target disease. For example, the antibodies disclosed herein may be administered in combination with chemotherapy, radiation therapy, and/or surgery for the treatment of cancer. An antibody administered “in combination” with another therapeutic agent does not have to be administered simultaneously with that agent. An antibody administered prior to or after another agent is considered to be administered “in combination” with that agent as the phrase is used herein. The antibodies may be administered by subcutaneous, peritoneal, intravascular, intramuscular, intradermal or transdermal injection, among other methods.

In certain embodiments, expression systems are provided for expressing the antibodies disclosed herein. These expression systems include polynucleotides encoding the antibodies, vectors comprising these polynucleotides, and host cells comprising these vectors. Polynucleotides encoding the antibodies may be isolated or synthesized using methods known in the art, and inserted into a replicable vector for amplification or cloning. Polynucleotides encoding variable light (V_{L}) and variable heavy (V_{H}) chains of the antibodies may be expressed from a single vector, or they may be expressed using two separate vectors, followed by in vitro assembly. In certain embodiments, they may be co-expressed from two separate vectors within the same cell and assembled intracellularly (see, e.g., U.S. Pat. No. 5,595,898). Suitable vectors may contain a variety of regulatory sequences, such as promoters, enhancers, or transcription initiation sequences, as well as genes encoding markers for phenotypic selection. Such additional sequences are well known in the art. In certain embodiments, the vector may contain a polynucleotide sequence encoding the constant regions of the heavy chain (C_{H}) and light chain (C_{L}) of a human IgG2 immunoglobulin. Alternatively, the vector may express only the V_{H} and V_{L} chains of the antibody, with the expressed polypeptide comprising an Fv fragment rather than a whole antibody. Vectors may be inserted into a suitable host cell for amplification or expression of the polynucleotide sequence. The host cells may be cultured for antibody production in a variety of media known in the art, such as for example Minimal Essential Medium (MEM) (Sigma), RPMI-1640 (Sigma), Dulbecco's Modified Eagle's Medium (DMEM) (Sigma), and Ham's F10 (Sigma). Media may be supplemented with a variety of agents, such as for example hormones, growth factors, salts, buffers, nucleotides, antibiotics, trace elements, glucose, or other energy sources. Culture conditions such as temperature and pH may be adjusted using parameters well known in the art.

The antibodies of this invention may comprise conjugates for specific delivery to cancer cells. In addition, binding of the antibodies to tumor cells may be used to recruit host immune responses. This host immune response may be increased by utilizing bivalent antibodies, with one binding site corresponding to the fully human antibodies provided herein and another binding site that recognizes cytotoxic T-cells.

In certain embodiments, the antibodies of the present invention may comprise oligosaccharides with high fucose content. In other embodiments, the antibodies of the present invention may have reduced fucose content, such as for example fucose-free Fc antibodies. Reduced fucose antibodies of the invention may be generated using a cell line with reduced fucosylation activity, such as for example rat YB2/0 cells (Shinkawa 2003) or the CHO variant cell line Lec13 (Shields 2002).

Pharmaceutical composition(s). Pharmaceutical compositions comprising an antibody or antigen-binding fragment thereof of the invention in association with a pharmaceutically acceptable carrier are also within the scope of the present invention (e.g., in a single composition or separately in a kit). The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

A pharmaceutical composition containing an antibody or antigen-binding fragment thereof of the invention, which is optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular, topical, intra-peritoneal, inhalation, intra-cranial) and non-parenteral (e.g., oral, transdermal, intranasal, intraocular, sublingual, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations may be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA (ethylenediaminetetraacetic acid) or EGTA (ethylene glycol tetraacetic acid). Pharmaceutical carriers may also include ethyl alcohol, polyethylene glycol and propylene glycol for water miscible vehicles; and sodium hydroxide, hydrochloric acid, citric acid or lactic acid for pH adjustment.

In an embodiment of the invention, preparations for parenteral administration may include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The concentration of the antibody or antigen-binding fragment thereof of the invention, which is optionally in association with a further chemotherapeutic agent, can be adjusted so that an injection provides an effective amount to produce the desired pharmacological effect. The exact dose depends, inter alfa, on the age, weight and condition of the patient or animal as is known in the art.

In an embodiment of the invention, unit-dose parenteral preparations are packaged in an ampoule, a vial or a syringe with a needle. All preparations for parenteral administration must be sterile, as is known and practiced in the art.

In an embodiment of the invention, a sterile, lyophilized powder is prepared by dissolving the antibody or antigen-binding fragment thereof, which is optionally in association with a further chemotherapeutic agent, or a pharmaceutically acceptable derivative thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-VEGF antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In an embodiment of the invention, for reconstitution, the lyophilized powder is added to sterile water or other liquid suitable carrier. The precise amount depends upon the selected therapy being given. Such amount can be empirically determined.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Generation of Antibodies that Bind hVEGF₁₆₅

Human single-chain Fv (scFv) phage display libraries were panned against immobilized hVEGF₁₆₅ to identify a panel of antibody fragments with the ability to bind hVEGF₁₆₅. Panning was carried out using standard protocols (see, e.g., Methods in Molecular Biology, vol. 178: Antibody Phage Display: Methods and Protocols Edited by: P. M. O'Brien and R. Aitken, Humana Press;, “Panning of Antibody Phage-Display Libraries,” Coomber, D. W. J., pp. 133-145, and “Selection of Antibodies Against Biotinylated Antigens,” Chames, P., et al., pp. 147-157).

Briefly, three wells of a NUNC® MAXISORP plate were coated with 50 μl of recombinant hVEGF₁₆₅ (R&D Systems, catalog no. 293-VE) at a concentration of 10 μg/ml in PBS. After overnight incubation at 4° C., free binding sites were blocked with 5% milk in PBS for one hour at room temperature. Approximately 200 μl of phage library in 5% milk/PBS was then added to the blocked wells and incubated at room temperature for approximately one to two hours. Wells were washed and bound phage was eluted using standard methods (see, e.g., Sambrook and Russell, Molecule Cloning: A Laboratory Manual, 3rd Edition, Cold Spring Harbor Laboratory Press, 2001). Eluted phage was amplified via infection for one hour at 37° C. into E. coli TG1 host cells in logarithmic growth phase. Infected TG1 cells were recovered by centrifugation at 2,500 RPM for five minutes, plated onto 15 cm 2YT-ampicillin-2% glucose agar plates, and incubated at 30° C. overnight. The panning process was then repeated using the amplified phage.

The cycle of panning, elution, and amplification was repeated for three rounds with decreasing concentration (e.g., 50 ug/ml hVEGF₁₆₅ at Round One, 10 ug/ml at Round Two, and then 10 ug/ml at Round Three), at which point single colonies from the plated TG1 cells were used to inoculate media in 96-well plates. Microcultures were grown to an OD₆₀₀ of 0.6, at which point expression of soluble scFv was induced by addition of 1 mM IPTG and overnight incubation in a shaker at 30° C. Bacteria were spun down, and periplasmic extract was used to test scFv binding to immobilized hVEGF₁₆₅ using a standard ELISA assay.

### Example 2

### Blocking of hVEGF₁₆₅ Binding to VEGF Receptors by XPA.10.064 and XPA.10.072 scFv

Phage clones from Example 1 exhibiting hVEGF₁₆₅ binding by were tested for their ability to block hVEGF₁₆₅ binding to VEGF-R1 and/or VEGF-R2 using the microplate-based competitive screening DELFIA® assay (Perkins Elmer, Waltham, Mass.).

Briefly, biotinylated hVEGF₁₆₅ solution was added 1:1 in volume to periplasmic extracts from Example 1 to a final concentration of 0.5 μg/ml. 100 μl of this mixture was added to a plate coated with VEGF-R1 or VEGF-R2 (R&D Systems: VEGF-R1/Flt-1, catalog no. 321-FL; VEGF-R2/KDR/Flk-1, catalog no. 357-KD) and incubated for 1.5 hours at room temperature. Plates were washed with PBST, and a 1:250 dilution of Europrium-Streptavidin in DELFIA Assay Buffer was added at 50 μl/well. Plates were incubated at room temperature for one hour, then washed with DELFIA Wash Buffer. DELFIA Enhancement Buffer was added at 50 μl/well, and plates were incubated for five minutes at room temperature. Plates were read on a Time-Resolved Fluorescence Gemini plate reader

### Example 3

### Conversion of scFv to scFv-Fc and IgG

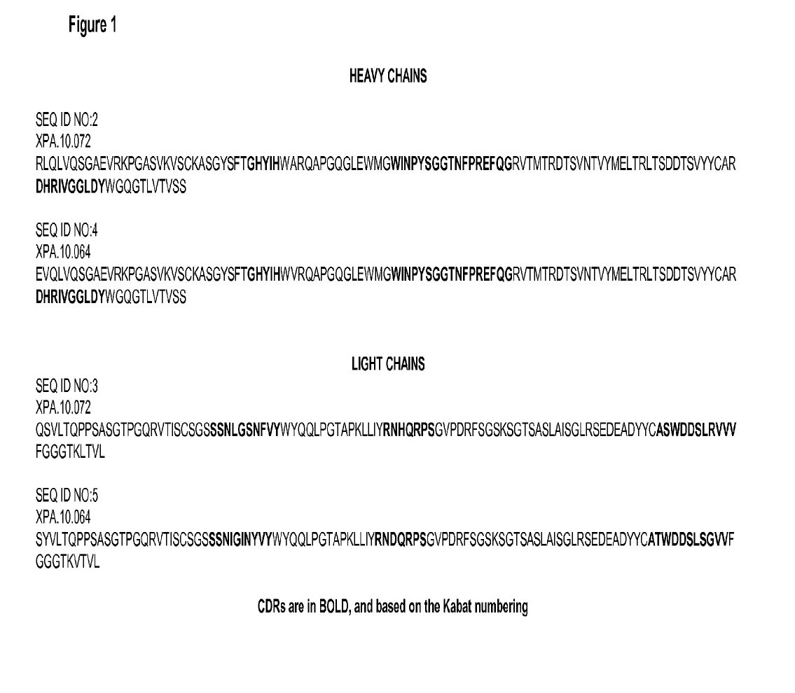

Two scFvs from Example 2 that inhibited hVEGF₁₆₅ binding to VEGF-R1 or VEGF-R2 by more than 60%, XPA.10.064 and XPA.10.072 were selected for conversion to scFv-Fc and/or IgG. The heavy chain variable regions (including heavy chain CDRs) and light chain variable regions (including light chain CDRs) of XPA.10.064 and XPA.10.072 are shown in FIG. 1. The heavy chain CDRs (e.g., HCDR1, HCDR2 and HCDR3) and the light chain CDRs (e.g., LCDR1, LCDR2, and LCDR3) are determined by the Kabat numbering system (Kabat, E. A., et al. 1987, in Sequences of Proteins of Immunological Interest, US Department of Health and Human Services, NIH, USA). The HCDR1, HCDR2 and HCDR3 for both XPA.10.064 and XPA.10.072 are determined to be amino acid sequences set forth in SEQ ID Nos. 6, 7, and 8 respectively. The LCDR1, LCDR2, and LCDR3 for XPA.10.064 are determined to be amino acid sequences set forth in SEQ ID Nos. 12, 13, and 14 respectively. The LCDR1, LCDR2, and LCDR3 for XPA.10.072 are determined to be amino acid sequences set forth in SEQ ID Nos. 9, 10, and 11 respectively.

For conversion of XPA.10.064 and XPA.10.072 into scFv-Fc fusion proteins, scFv cDNAs were cloned into eukaryotic expression vectors that had been modified to encode the CH2 and CH3 domains of the gamma-2 (γ2) heavy chain constant region gene (U.S. Pat. No. 7,192,737; WO 2004/033693).

For conversion of XPA.10.064 and XPA.10.072 into IgG, the variable regions of both heavy and light chains were cloned into eukaryotic expression vectors encoding the kappa (κ), lambda (λ), or gamma-2 (γ2) heavy and light chain constant region genes (US 2006/0121604).

XPA.10.064 and XPA.10.072 scFv-Fc and IgG antibodies were transiently expressed in 293E cells as described previously (US 2006/0121604). Supernatant from transfected cells was harvested at day six of culture, and IgG was purified by Protein-A chromatorgraphy.

### Example 4

### Biacore Analysis of XPA.10.064 and XPA.10.072 scFv-Fc and IgG Binding Kinetics

Binding affinity of XPA.10.064 and XPA.10.072 scFv-Fcs was assessed using a BIACORE 2000 and a CM5 sensor chip (Biacore) with Protein AIG (Piece) immobilized on all flow cells at high density. Dilution and running buffer for these experiments was HBS-EP (Biacore) with 1:50 dilution of Chemiblocker® (Chemicon). Antibody capture was performed by injecting diluted XPA.10.064 and XPA.10.072 scFv-Fcs over flow cell 2 (fc2) at 20 μl/minute for a variable volume to achieve roughly 50-70 RU of antibody capture. Antibody concentrations were approximately 0.5 μg/ml. hVEGF₁₆₅ expressed from sf21 cells was injected over five minutes at 30 μl/minute using the Kinject feature with 15 minute dissociation over fc1 and 2. Four dilutions of hVEGF₁₆₅ were prepared in a three-fold serial dilution, giving concentrations of 5 μg/ml (119 nM), 1.667 μg/ml (39.7 nM), 0.55 μg/ml (13.2 nM), and 0.185 μg/ml (4.4 nM). Regenerations were performed with two injections of 100 mM HCl at 50 μl/minutes for twelve seconds each. Data was processed in Scrubber2 and fit by a 1:1 Langmuir interaction model after double referencing of the control flow cell and blank injections. XPA.10.064 and XPA.10.072 scFv-Fcs exhibited high and nearly identical binding affinity for hVEGF₁₆₅.

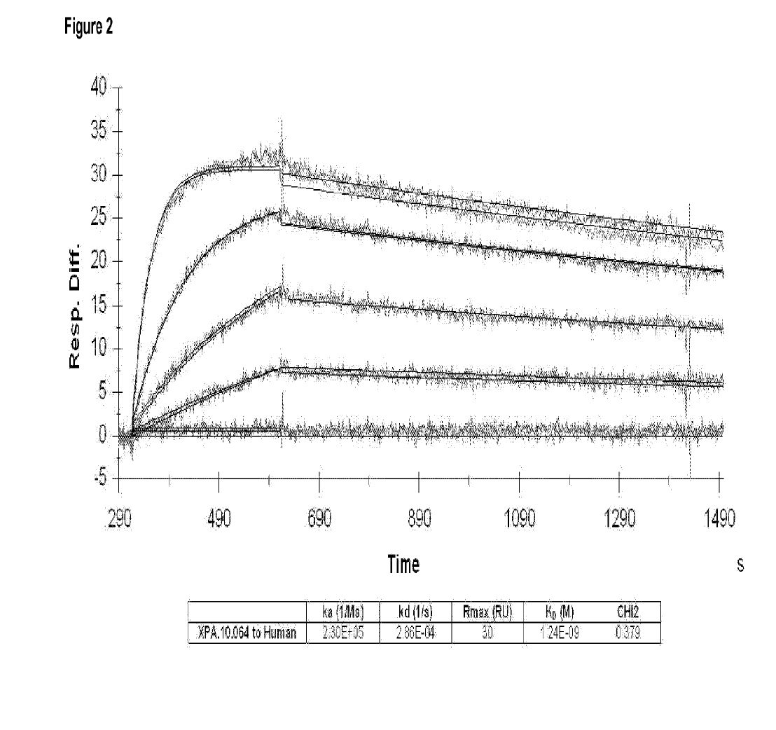

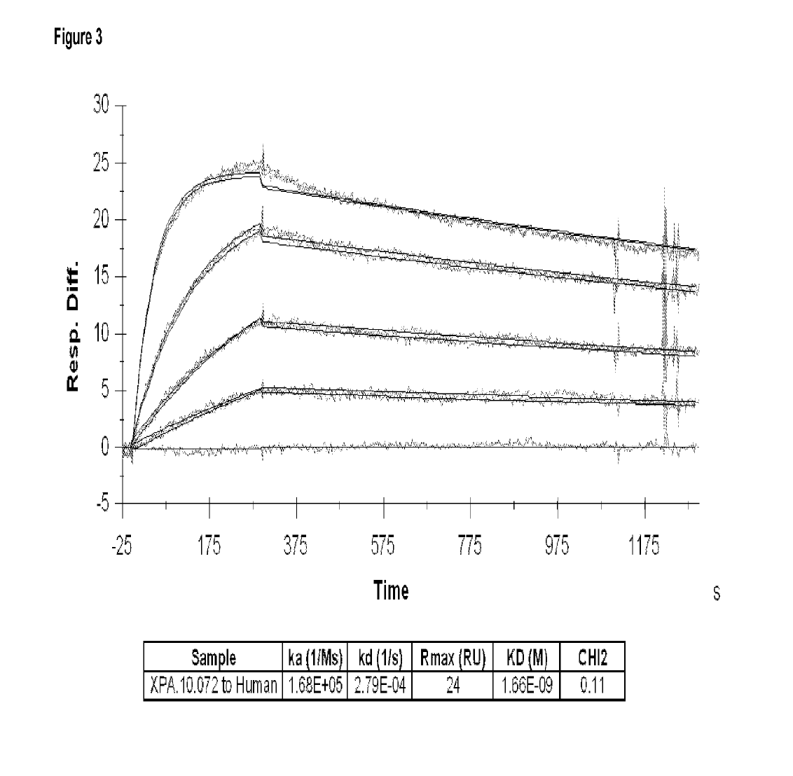

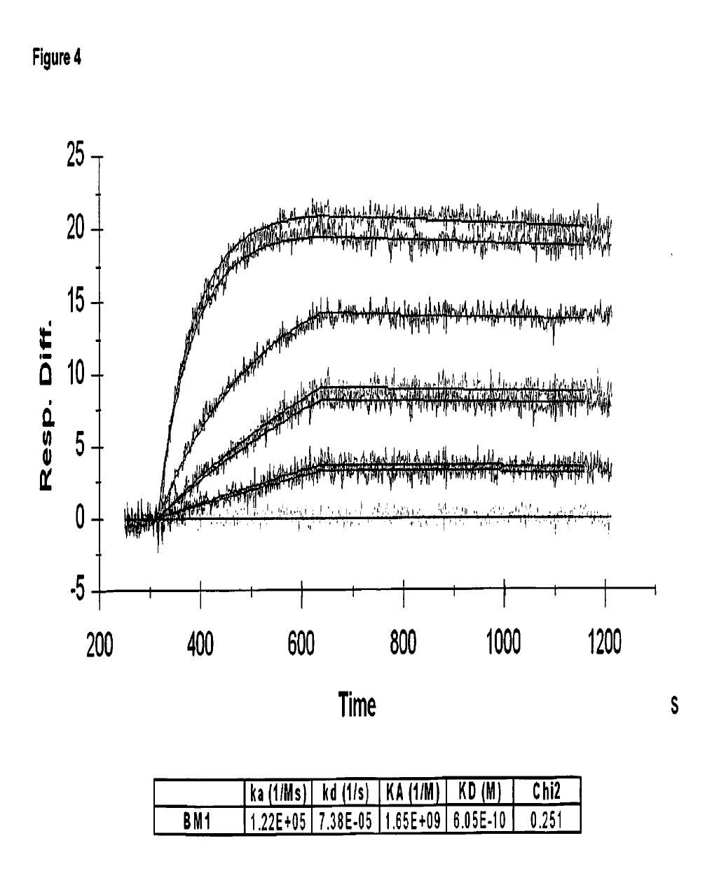

A similar protocol was utilized to assess binding kinetics of XPA.10.064 and XPA.10.072 IgG2. Both XPA.10.064 and XPA.10.072 IgG2s bound specifically to hVEGF₁₆₅ with similar low single digit nanomolar affinity (FIGS. 2-4), and exhibited only weak binding to mVEGF₁₆₅. Both antibodies exhibited a kₐ value similar to that of Bevacizumab (Table 2).

### Example 5

### Blocking of hVEGF₁₆₅ Binding to VEGF Receptors by XPA.10.064 and XPA.10.072 IgGs

The ability of XPA.10.064 and XPA.10.072 IgG2s to block binding of hVEGF₁₆₅ to VEGF-R1 and/or VEGF-R2 was assessed using a Biacore 2000 with a CM5 chip.

VEGF receptor (R&D Systems) was immobilized on the CM5 chip at a density of approximately 15,000 via amine coupling (Biacore). VEGF-R2 was immobilized on fc2 and VEGF-R1 was immobilized on fc4. Flow cells 1 and 3 served as references, and were activated and blocked in the same manner as the receptor immobilized flow cells. 0.15 μg/ml hVEGF₁₆₅ in HBS-EP running buffer was mixed 1:1 with antibody sample or buffer. Final antibody concentrations were 15, 5, 1.667, 0.556, 0.185, 0.0617, 0.0206, and 0 μg/ml. Samples were incubated for at least one hour prior to initiation of the Biacore analysis run. All samples were injected in duplicate and each set of antibody replicates had its own positive and negative control (no antibody with VEGF and no VEGF, respectively). Samples were injected at 10 μl/minute for 1.5 minutes over all flow cells. Regeneration was performed with a twelve second injection of Glycene, pH 1.75, at 50 μl/minute.

For data analysis, the slope of a linear portion of association (30 seconds to one minute) of the association phase was determined with a linear fit. The signal from each point was subtracted by the nearest blank, then divided by the matched 100% signal (no antibody) control to give the percent inhibition of that cycle. Data was plotted in GraphPad Prism and fit with a sigmoidal dose response curve to calculate the EC50.

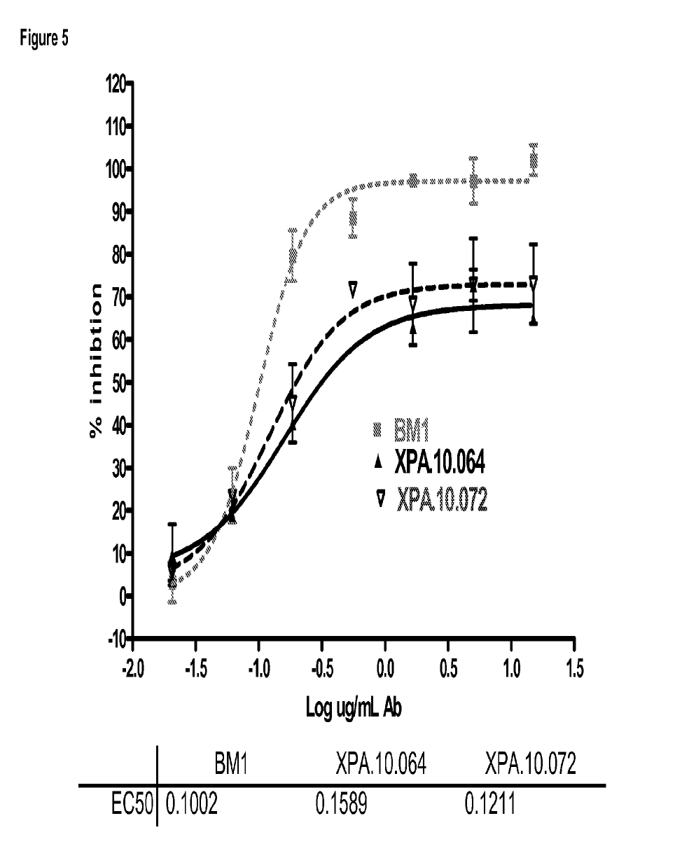

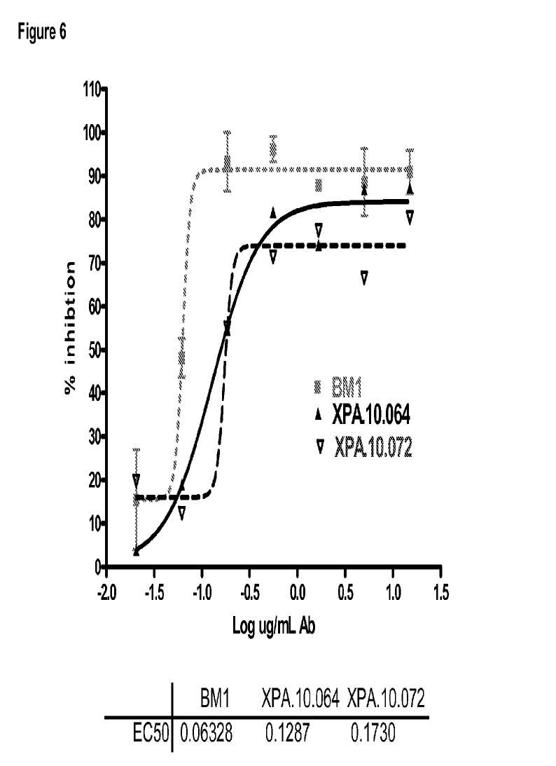

XPA.10.064 and XPA.10.072 IgG2s both blocked hVEGF₁₆₅ binding to VEGF-R1 and VEGF-R2 at levels similar to Bevacizumab (FIG. 5-6, Table 3).

TABLE 3

| | Bevacizumab | XPA.10.064 | XPA.10.072 |
|---|---|---|---|
| hVEGF₁₆₅/VEGF-R1 | 0.1002 | 0.1589 | 0.1211 |
| (EC50) | | | |
| hVEGF₁₆₅/VEGF-R2 | 0.06328 | 0.1287 | 0.1730 |
| (EC50) | | | |

### Example 6

### Analysis of hVEGF₁₆₅ Epitopes Bound b XPA.10.064 and XPA.10.072

To determine whether the hVEGF₁₆₅ epitopes recognized by XPA.10.064 and XPA.10.072 were linear or conformational, three 200 ng samples of non-reduced or reduced and heat-denatured recombinant hVEGF₁₆₅ were subjected to electrophoresis on three separate SDS-PAGE gels. Electrophoresed proteins were transferred to Immulon-P membranes, and the blots were hybridized with XPA.10.064 IgG, XPA.10.072 IgG or Bevacizumab antibodies and incubated with 1 μg/ml secondary goat anti-human IgG HRP-conjugated antibody. Binding was detected with enhanced chemiluminescence (ECL) substrate (Pierce).

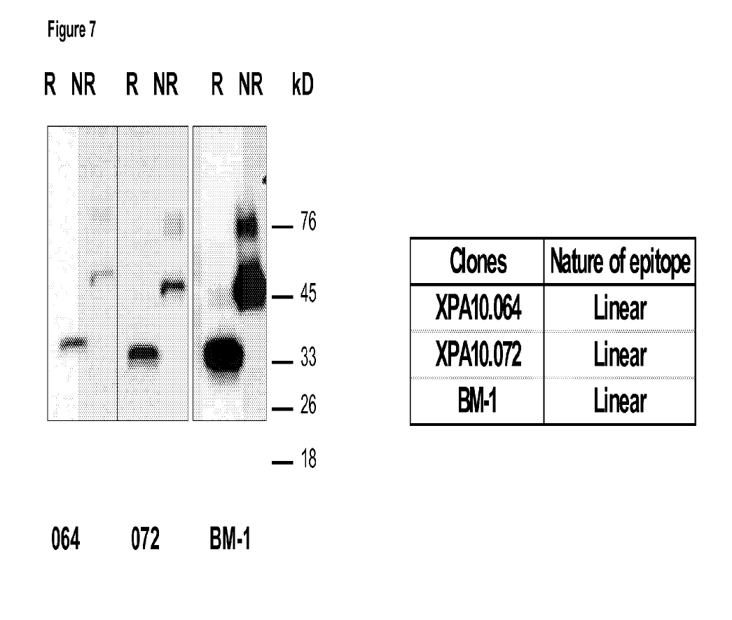

XPA.10.064, XPA.10.072, and Bevacizumab all bind to linear epitopes on hVEGF₁₆₅ (FIG. 7).

### Example 7

### XPA.10.064 and XPA.10.072 hVEGF₁₂₁ Epitope Binding Studies

To determine whether XPA.10.064 and XPA.10.072 bind the same hVEGF₁₂₁ epitope as Bevacizumab, three ELISA comparative binding assays were performed using various hVEGF₁₂₁ mutants.

Previous mutation analysis has shown that VEGF residues M81, G88, Q89, and G92 are important for Bevacizumab binding to hVEGF₁₆₅ (Fuh 2006). To determine whether these residues are important for XPA.10.064 and XPA.10.072 binding, the following hVEGF₁₂₁ mutants were generated: hVEGF₁₂₁-M81A, hVEGF₁₂₁-Q89A, hVEGF₁₂₁-G92A, and hVEGF₁₂₁-G88S. Mutants were transiently expressed in CHO-K1 cells, and cell supernatants were collected for binding analysis by ELISA.

A microtiter plate was coated with XPA.10.064, XPA.10.072, Bevacizumab, or a control polyclonal goat anti-human VEGF (PAB) at a concentration of 1, 2, or 5 μg/ml and incubated overnight at 4° C. The plate was blocked with 30% ChemiBlock™ reagent (Millipore) in PBS for one hour at room temperature, and 30, 60, or 100 μl of CHO-K1 culture supernatant for each mutant, or 1 μg/ml of wild-type hVEGF₁₂₁, recombinant hVEGF₁₆₅, or recombinant mVEGF₁₆₅, was added to appropriate wells. After one hour of incubation, the plate was washed and incubated with a biotinylated goat polyclonal anti-VEGF antibody for one hour at room temperature. Detection was performed with HRP-conjugated streptavidin, followed by TMB chromogenic substrate (Calbiochem) using manufacturer protocol.

The binding pattern of XPA.10.064 and XPA.10.072 to hVEGF₁₂₁ mutants was similar to that of Bevacizumab (Tables 4-5), indicating that the antibodies bind overlapping or similar epitopes.

### Example 8

### Tissue Cross-Reactivity of XPA.10.064 and XPA.10.072

A frozen normal human tissue array (TMA) was used to evaluate immunohistochemical (IHC) reactivity of XPA.10.064 and XPA.10.072 using a single-color chromogenic technique. The TMA comprised 32 normal human tissue types, with each type consisting of tissues from two to three different donors. In addition to the TMA, larger sections of normal human liver, kidney, Fallopian tubes, pancreas, ureter, and adrenal gland were used to confirm statining results from the TMA or to replace missing tissues in the TMA. Positive controls included hVEGF proteins spotted on UV-resin slides and renal carincoma tissue expressing high level hVEGF as assessed by strong staining with anti-hVEGF rabbit monoclonal antibody.

The TMA and normal human tissues and the hVEGF protein spot and renal carcinoma positive controls were stained with XPA.10.064, XPA.10.072 (human IgG2), or Bevacizumab at 20 μg/ml using a human-on-human IHC staining protocol. A human tonsillitis case was also included to monitor the effectiveness of the staining protocol. The final protocol did not have reactivity with tissue endogenous immunoglobulin in B-cell region of the tonsil tissue. Negative control antibodies were human IgG1 and IgG2 (Sigma, St. Louis, Mo.) and the human KLH antibody CHO.KLHG2.60 (IgG2).

XPA.10.064, XPA.10.072, and Bevacizumab were all reactive with hVEGF protein spots at 2-3+ on a scale of 0-4+, where 4+ indicates the highest staining intensity. For renal carcinoma tissue, Bevacizumab gave equivocal staining, while XPA.10.064 and XPA.10.072 stained cytoplasm of tumor cells.

Human IgG1 and IgG2 did not stain any tissue elements, giving only minimal background staining. CHO.KLHG2.60 had reactivity with cells in the adrenal cortex, epithelial cells in esophagus, mammary gland, pancreas, prostate, stomach, thyroid gland, ureter cervix, and Fallopian tube. Due to this reactivity, human IgG1 and IgG2 were used as reference negative controls.

XPA.10.064 had the broadest tissue reactivity spectrum of the test antibodies. XPA.10.064 stained strongly with smooth muscle cells in bladder, GI track, Fallopian tube, mammary gland, prostate, ureter, and uterus, and with epithelial cells in Fallopian tube, prostate, skin, small intestine, stomach, thyroid gland, ureter, endometrial glands of the uterus, and uterus cervix. In addition, XPA.10.064 stained some neurons and nerve fibers in cerebellum, cerebral cortex, and spinal cord, as well as cardiac and skeletal muscles, cells in pituitary glands, renal glomeruli, liver sinusoid endothelium, stromal cells of thymus, macrophages in lung, and cells in the adrenal cortex. XPA.10.072 stained strongly with nerve fibers in cerebellum, cerebral cortex, and spinal cord. XPA.10.072 also stained smooth muscles of the GI track, Fallopian tube, prostate, ureter, and uterus, epithelial cells in esophagus, Fallopian tube, mammary gland, prostate, stomach, small intestine, thyroid gland, and ureter, macrophages in lung, and fibroblast/histiocytes in placenta. Bevacizumab stained negative with all normal human tissues.

To determine whether the immunohistochemical reactivity of XPA.10.064 and XPA.10.072 represented on- or off-target binding, a multicolor immunofluorescence-based approach was utilized. This approach is based on the simultaneous comparison of immunoreactivity of a known “gold standard” anti-VEGF antibody to test antibodies. On-target reactivity of test antibodies manifests as co-localization with the “gold standard” antibody, while lack of co-localization indicates off-target reactivity.

Frozen sections of cell pellets from positive (Du145) and negative (Hek 293) control cells were stained with commercial mouse anti-human anti-VEGF antibody (BD Pharmingen, clone G153-694) using the same chromogenic IHC methodology employed in the initial experiments. This antibody stained Du145 cells, but failed to stain Hek 293 cells. In addition, frozen sections of colon carcinoma stained with this antibody demonstrated a characteristic pattern of reactivity in the epithelial and tumor associated matrix components with good internal negative controls. Therefore, G153-694 was designated the “gold standard” positive control antibody.

Using a protocol from the Zenon IgG Labeling Kit (Molecular Probes), primary antibody was pre-incubated with a fluorochrome conjugated Fc-targeted anti-human Fab, followed by neutralization of non-reacted Fab with molar excess of the appropriate normal serum. Fluorescent antibody-Fab complex used as the staining reagent, followed by nuclear counterstaining with DAPI. Frozen sections from an adenocarcinoma of the colon (Tissue ID 4558) were used as control VEGF-positive tissue for these co-localization studies. XPA.10.064 and XPA.10.072 antibodies were labeled with red (Alexa Fluor 594), and “gold standard” antibody was labeled with green (Alexa Fluor 488). The assay was repeated using the reverse color combination, which gave essentially the same results. Images were captured using a Leica TCS-SP, model DM RXE laser scanning confocal microscope and Leica Confocal software, version 2.0 (Leica Microsystems, Wetzler, Germany). Multiple fields were imaged at 400× (at least three), and representative fields were analyzed for colocalization using Image Pro software (Media Cybernetics, Silver Spring, Md.). Since Bevacizumab was essentially negative in all of the initial IHC studies on both colon carcinoma and frozen pellet sections, it was not included in the co-localization studies.

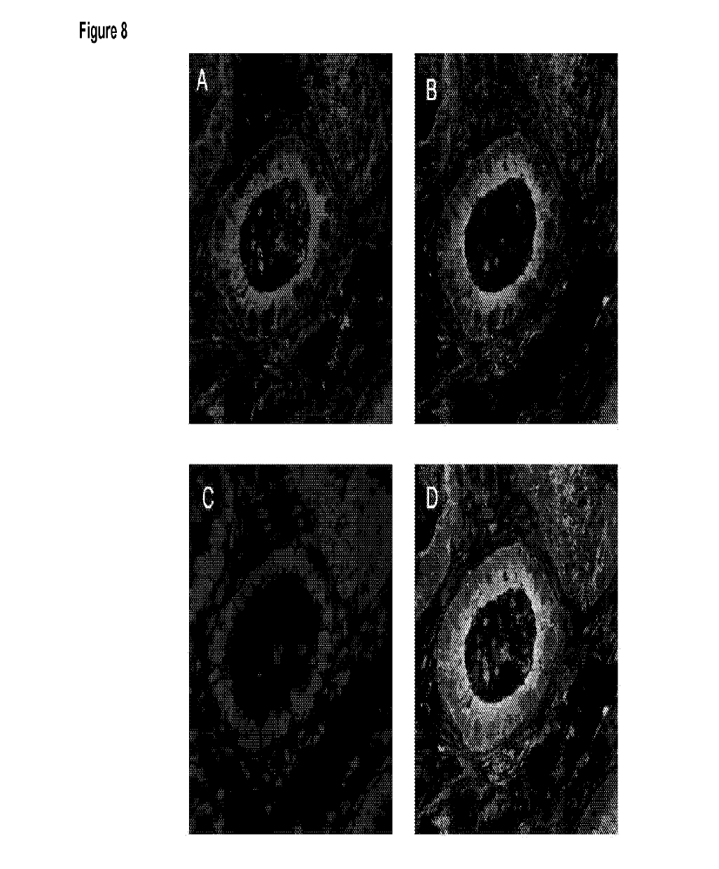

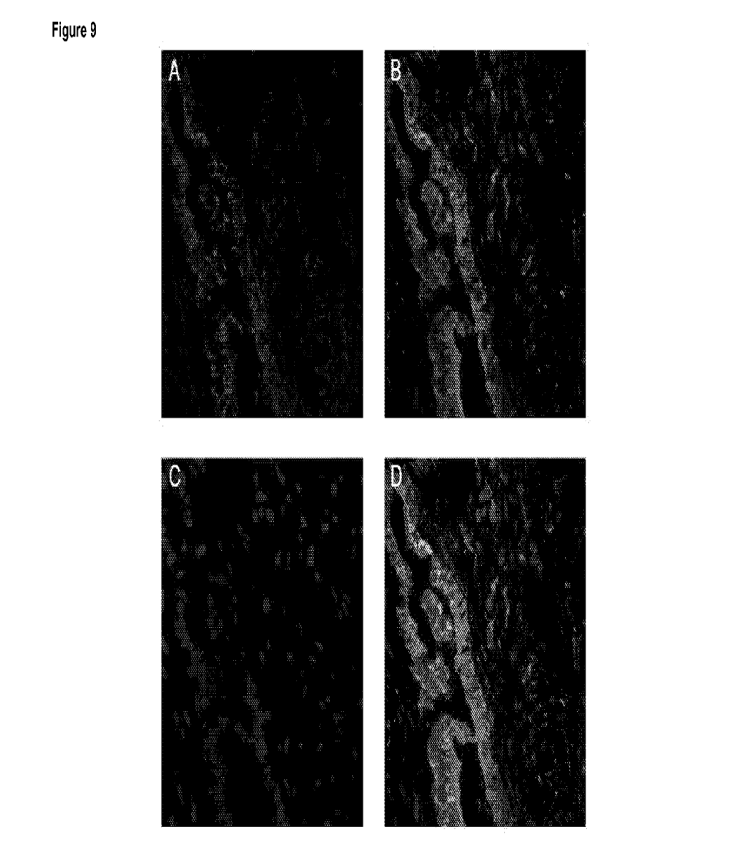

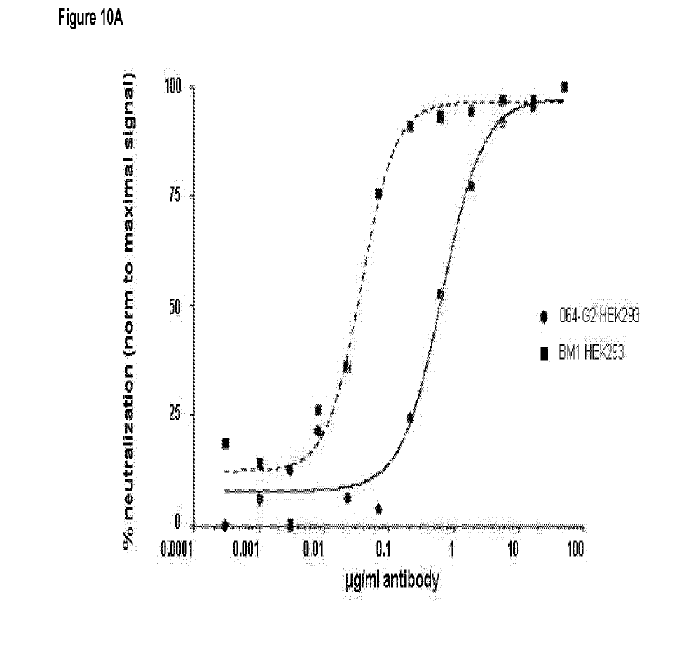

As anticipated based on the initial IHC experiments, XPA.10.064 and XPA.10.072 demonstrated similar high degree of colocalization, with XPA.10.064 showing the greatest intensity of tissue staining (FIGS. 8-9).

### Example 9

### Inhibition of HUVEC Proliferation by XPA.10.064 and XPA.10.072

XPA.10.064 and XPA.10.072 scFv-Fcs and IgG2s were tested for their ability to block proliferation of HUVECs.

Pooled HUVECs (Clonetics #CC-2519) were grown in ECGM Complete Media (Clonetics #CC-3024) plus BulletKit-2 (supplemented with rhEGF, rhFGF, rhVGEF, ascorbic acid, hydrocortisone, IGF, heparin, gentamycin/amphotericin, and 2% FBS). Cells were seeded at 2-3×10⁵ cells per T-75 flask, and reached confluence at 3-4 days. The sub-confluent monolayer was washed with PBS, trypsinized, and neutralized with complete media containing PBS.

To measure HUVEC proliferation in the presence of hVEGF₁₆₅, a 16-point dose titration of hVEGF₁₆₅ expressed from HEK 293 cells was set up by diluting in basal growth medium (0-200 ng/ml final, 2× dilutions, 2× concentration, 50 μl/well). HUVEC cells were re-suspended at 2×10⁵ cells/ml in cold basal medium/0.1% BSA, and 50 μl of cells (1×10⁴ c/w) were added to each well of the hVEGF₁₆₅ titration plate for a final volume of 100 μl/well. Outer wells were flooded with PBS, and plates were sealed with parafilm to prevent dehydration. Plates were incubated for 96 hours in 5% CO₂ at 37° C., then brought to room temperature over approximately 15-20 minutes. Cell TiterGlo (TTG, Promega) was thawed and brought to substrate temperature, and 100 μl of substrate/buffer mixture was added to each well. The plate was shaken on an orbital plate shaker for 1-2 minutes, and 150 μl from each well was transferred to white bottom, white walled plates and incubated in the dark for 5-10 minutes. The plate was read on a luminometer with a one second integration.

For the proliferation inhibition assay, titrations of XPA.10.064, XPA.10.072, and Bevacizumab were generated (0-50 μg/ml final, 3× dilutions, 4× concentrations, 25 μl/well final volume). 1:1 hVEGF₁₆₅: antibody was added at 4× concentrations for both growth factor and antibody, and the plate was incubated for two hours in 5% CO₂ at 37° C. 50 μl/well of VEGF/antibody complex was added to 50 μl/well of re-suspended HUVEC cells, and the plates were incubated for 96 hours and treated with TiterGlo buffer as described above.

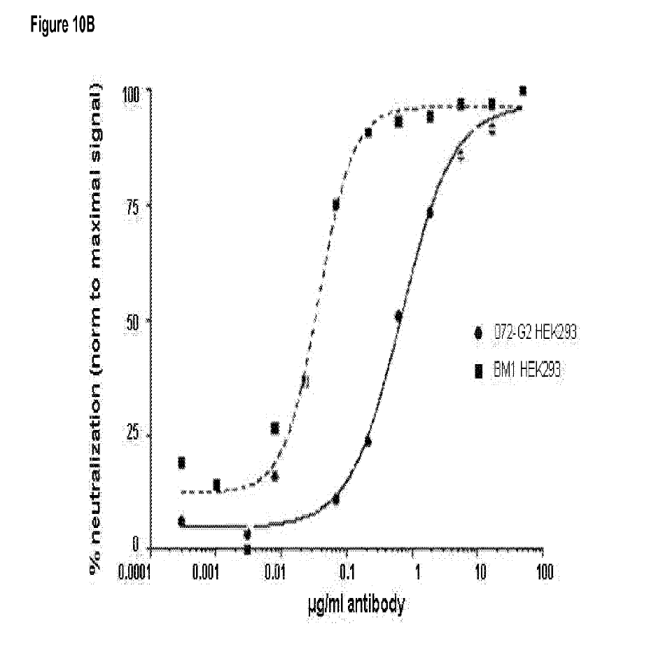

XPA.10.064 and XPA.10.072 scFvs and IgG2s inhibited HUVEC proliferation. IgG2 results are set forth in FIG. 10 and Table 6).

TABLE 6
Inhibition of HUVEC proliferation by XPA.10.064,
XPA.10.072, and Bevacizumab.

| | EC50 (μg/ml) | | |
|---|---|---|---|
| | XPA.10.064 | XPA.10.072 | BM-1 |
| HEK 293 hVEGF₁₆₅ | 0.66 | 0.68 | 0.04 |

### Example 10

### Inhibition of VEGF-R2 Phosphorylation by XPA.10.064 and XPA.10.072

The ability of XPA.10.064 and XPA.10.072 to inhibit VEGF-R2 phosphorylation by hVEGF₁₆₅ was analyzed by ELISA.

To generate lysate plates, HUVEC cells between passages two and six were thawed and plated into TC flasks in EGM2 complete media (Lonza), and allowed to grow for one to two passages. Sub-confluent cells were trypsinized, neutralized with complete media, washed twice with PBS, and counted. Cells were plated at 1×10⁵ cells/well in complete media in 24w format (triplicate wells) and incubated at 37° C. for 24 hours. After incubation, cells were washed twice with room temperature PBS and starved in EBM2 medium (Lonza) plus 0.1% BSA for five hours. PBS was decanted and cells were incubated with a dose titration of hVEGF₁₆₅ (stimulation) or pre-complexed VPA.10.064+hVEGF₁₆₅, VPA.10.072+hVEGF₁₆₅, or Bevacizumab+hVEGF₁₆₅ (inhibition) for five minutes. VPA.10.064+hVEGF₁₆₅ and VPA.10.072+hVEGF₁₆₅ were generated by mixing a 2× dose titration of antibody 1:1 with 2× hVEGF₁₆₅ (final concentration: 20 ng/ml) and incubating at 37° C. for 24 hours. hVEGF₁₆₅, VPA.10.064+hVEGF₁₆₅, and VPA.10.072+hVEGF₁₆₅ were decanted and cells were washed twice with ice cold PBS. 65 μl/well of lysis buffer/well (1% NP-40, 20 mM Tris, pH 8.0, 137 mM NaCl, 10% glycerol, 2 mM EDTA, 1 mM activated sodium orthovanadate, 10 μg/ml Leupeptin) was added, and cells were rocked at 4° C. for 30 minutes until needed.

Capture antibody specific for VEGF-R2 (R&D Systems, VEGF-R2/KDR/Flk-1, catalog no. 357-KD) was diluted to a working concentration of 8.0 g/ml in PBS and coated onto a 96 well microplate at 100 μl/well. VEGF-R2/KDR/Flk-1 binds both phosphorylated and non-phosphorylated VEGF-R2. The plate was sealed and incubated overnight. Each well was aspirated and washed with wash buffer five times, and the plate was blocked by adding 300 μl/well of block buffer and incubating at room temperature for one to two hours. Each well was aspirated and washed with wash buffer five more times, and 100 μl of HUVEC lysate was added to each. The plate was incubated for two hours at room temperature, and wells were aspirated and washed five times with wash buffer. 100 μl of HRP-conjugated detection antibody specific for phsophorylated tyrosine was added to each well, and the plate was covered and incubated for two hours at room temperature out of direct light. Wells were aspirated and washed five times with wash buffer, and 100 μl of substrate solution was added to each well. The plate was incubated for 20 minutes at room temperature out of direct light, and 50 μl of stop solution was added to each well. The optical density of each well was read on a microplate reader at 450 nm.

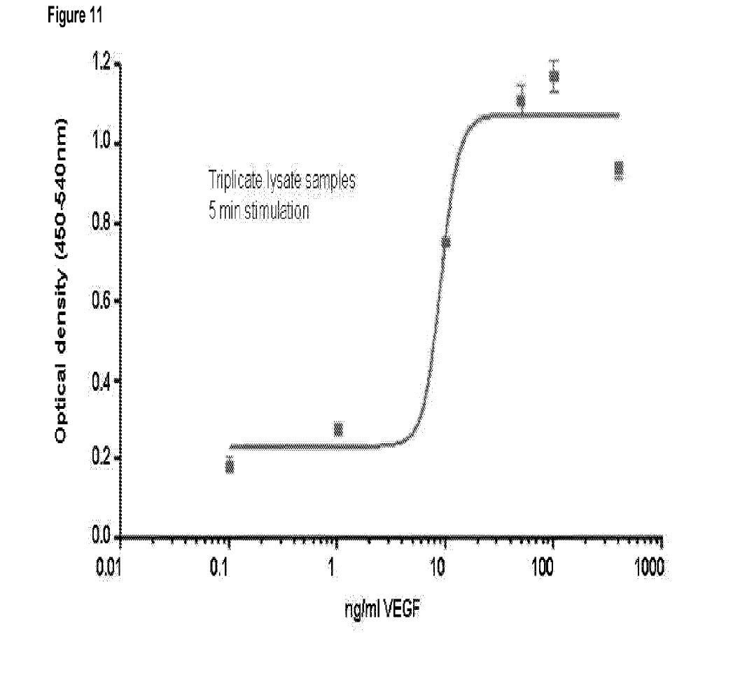

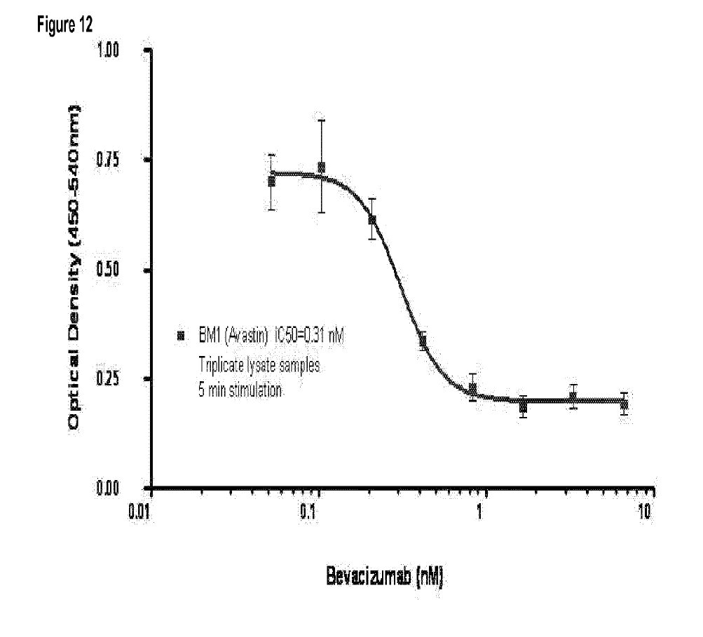

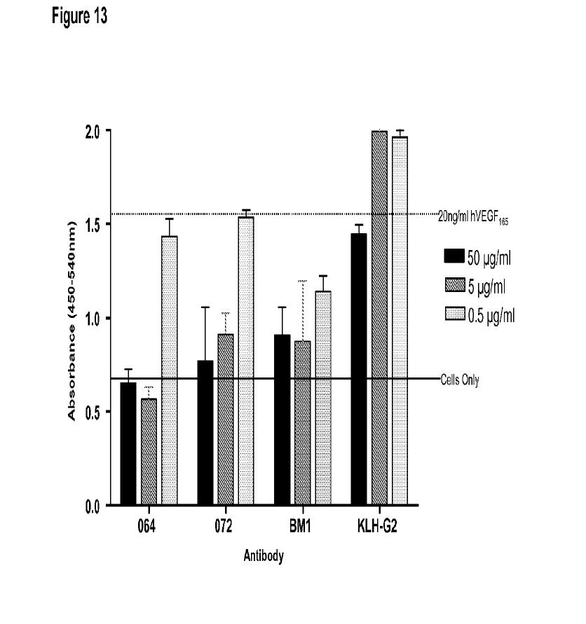

HUVECs treated with a dose titration of hVEGF₁₆₅ exhibited an increase in phosphorylated VEGF-R2 (FIG. 11). HUVECs treated with a dose titration of Bevacizumab+hVEGF₁₆₅ exhibited a decrease in VEGF-R2 phosphorylation (FIG. 12). HUVECs treated with a dose titration of XPA.10.064+hVEGF₁₆₅ or XPA.10.072+hVEGF₁₆₅ exhibited a decrease in VEGF-R2 phosphorylation. Results for each antibody are summarized in FIG. 13.

### Example 11

### Inhibition of Angiogenesis by XPA.10.064 and XPA.10.072

A Matrigel plug assay was used to measure the ability of XPA.10.064 and XPA.10.072 to inhibit angiogenesis in vivo.

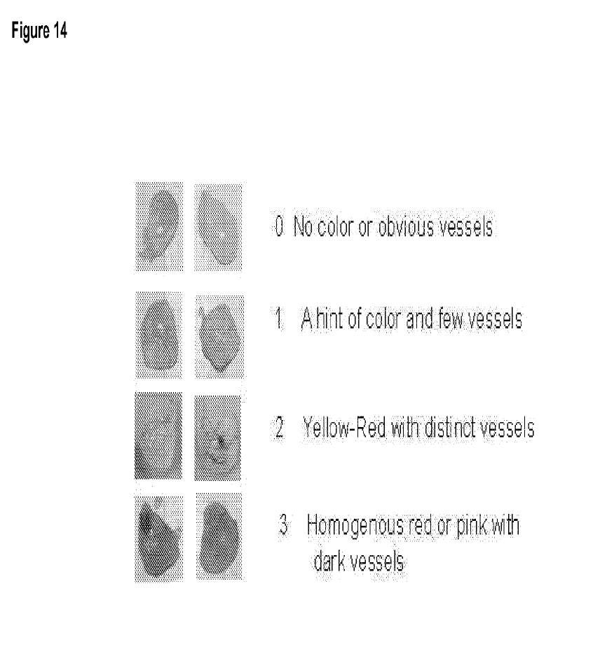

Female NU/NU mice age 6-7 weeks were injected s.c. in the abdomen with 0.5 ml Matrigel containing 2×10⁶ DU145 cells, which produce human VEGF to induce angiogenesis. Mice were injected i.p. on days 0 and 3 with vehicle control or 0.1, 1, or 5 mg/kg XPA.10.064, XPA.10.072, or Bevacizumab. On day 7, mice were sacrificed and Matrigel plugs were excised, weighed, and photographed. Plugs were given a visual score of 0 to 3 based on the following scheme: 0, no color or obvious vessels; 1, hint of color and few vessels; 2, yellow-red with distinct vessels; and 3, homogenous red or pink with dark vessels (FIG. 14). Plugs were evaluated by blinded scorers who received photographs in which the plug order was scrambled.

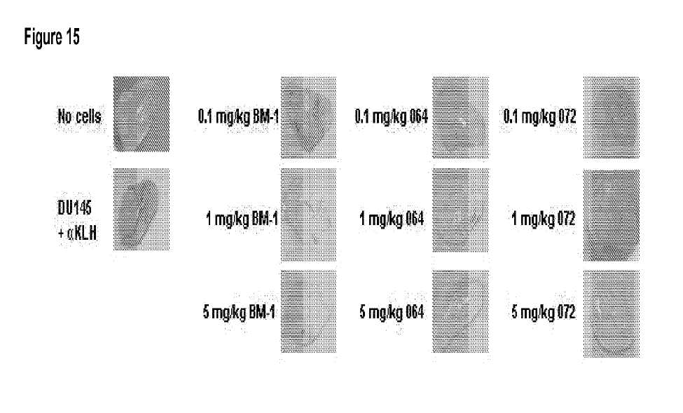

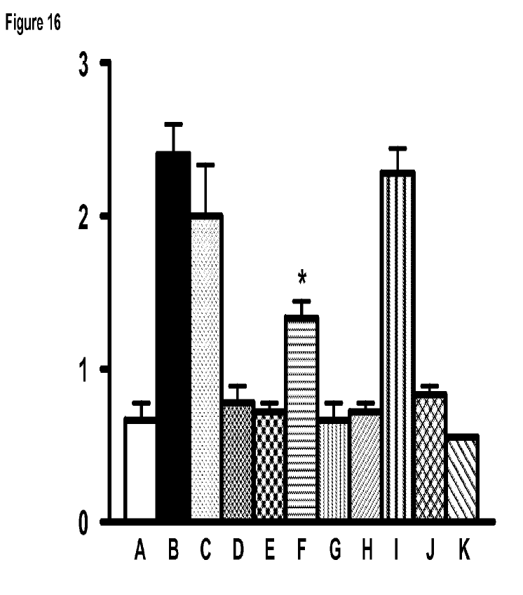

Administration of XPA.10.064 resulted in a significant decrease in angiogenesis at all dosages tested, while administration of XPA.10.072 resulted in a significant decrease at 1 mg/kg and 5 mg/kg (FIGS. 15 &16). The level of angiogenesis inhibition was similar to that observed in the presence of Bevacizumab.

XPA.10.064, XPA.10.072, and Bevacizumab concentrations in mouse serum were measured by ELISA at four days after the last antibody dose. There was no significant difference in antibody levels between the three antibodies at any of the dosages tested.

### Example 12

### Inhibition of Tumor Growth by XPA.10.064 and XPA.10.072

The ability of XPA.10.064 and XPA.10.072 to inhibit tumor growth was tested with the A673 Rahbdomyosarcoma tumor growth model using a previously disclosed protocol (Liang 2006). A673 cells maintained in culture were grown until confluent, then harvested and re-suspended in sterile Matrigel. Xenografts were established by s.c. injection of 5×10⁶ cells in Matrigel into the flanks of six-week-old female nude mice. When tumor size reached about 100 mm³, mice were randomized into eight groups of ten and injected i.p. with vehicle only (Group 1), 0.5 mg/kg XPA.10.064 IgG2 (Group 2), 5 mg/kg XPA.10.064 IgG2 (Group 3), 0.5 mg/kg XPA.10.072 IgG2 (Group 4), 5 mg/kg XPA.10.072 IgG2 (Group 5), 5 mg/kg CHO.KLH IgG2 (Group 6), 0.5 mg/kg Bevacizumab (Group 7), or 5 mg/kg Bevacizumab (Group 8) twice a week for 18 days (six total doses). Blood and tissue samples were collected at 24, 72, and 168 hours after the last dose.

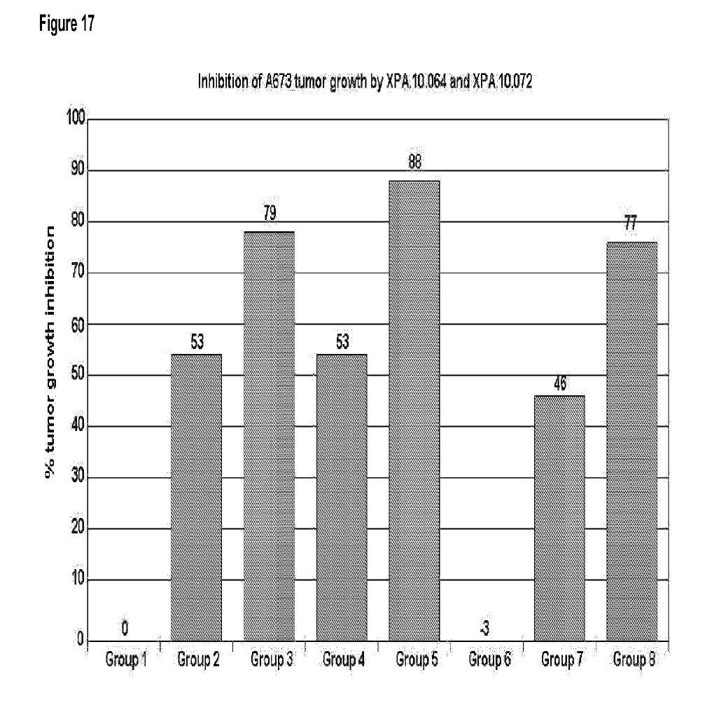

Administration of XPA.10.064 and XPA.10.072 resulted in significant in vivo tumor growth inhibition at both dosages tested (FIG. 17). The level of growth inhibition for both antibodies was slightly higher than that observed with Bevacizumab at all dosages tested. Serum levels were similar for all antibodies administered at a given dosage (0.5 mg/kg, serum level approximately 5-7 μg/ml; 5 mg/kg, serum level approximately 75-100 μg/ml).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference in their entirety as if fully set forth herein.

### REFERENCES

1. Amoroso, A., et al. 1997. Eur Rev Med Pharmacol Sci 1:17-25.
2. Azzazy, H. E., Highsmith, W. E. Jr. 2002. Clin Biochem 35:425-445.
3. Berkman, R. A., et al. 1993. J Clin Invest 91:153-159.
4. Brown, L. F., et al. 1993. Cancer Res 53:4727-4735.
5. Brown, L. F., et al. 1995. Human Pathol 26:86-91.
6. Cumbers, S. J., et al. 2002. Nat Biotechnol 20:1129-1134.
7. Dimmeler, S., Dernbach, E., Zeiher, A. M. 2000. FEBS Lett 477:258-262.
8. Dupont, J., et al. 2005. Proc Am Soc Clin Oncol 23:199s.
9. Dvorak, H. F., et al. 1995. Am J Pathol 146:1029-1039.
10. Erikkson, U., Alitalo, K. 1999. Curr Top Microbial Immunol 237:41-57.
11. Ferrara, N. 1999. Curr Top Microbiol Immunol 237:1-30.
12. Ferrara, N., et al. 2004. Nat Rev Drug Discov 3:391-395.
13. Fishwild, D. M., et al. 1996. Nat Biotechnol 14:845-851.
14. Fuh, G., et al. 2006. J Biol Chem 281:6625-6631.
15. Giri, J. G., et al. 1994. EMBO J 13:2822-2830.
16. Harris, A. L. 2000. Oncologist 5 Suppl 1:32-36.
17. Hawkins, R. E., Russell, S. J., Winter, G. 1992. J. Mol Biol. 226:889-896.
18. Holash, J., et al. 2002. Proc Natl Acad Sci USA 99:11393-11398.
19. Houston, et al. 1988. Proc Natl Acad Sci USA 85:5879-5883.
20. Hunt, S. 2001. Curr Opin Mol Ther 3:418-424.
21. Jianhua, H., Kontos, C. D. 2002. J Biol Chem 277:10760-10766.
22. Jostock, T., et al. 2004. J Immunol Methods 289:65-80.
23. Katre, N. V. 1990. J Immunol 144:209-213.
24. Khamaisi, M., et al. 2003. Nephrol Dial Transplant 18:1427-1430.
25. Kim, K. S., et al. 2003. J. Biol Chem 278:11449.
26. Lee, Y. K., et al. 2004. Blood 104:788-794.
27. Li, J., et al. 2006. Proc Natl Acad Sci USA 103:3557-3562.
28. Liang, W-C, et al. 2006. J Biol Chem 281:951-961.
29. Lonberg, N. 2005. Nat Biotechnol 23:1117-1125
30. Lowman, H. B. 1998. Phage display of peptide libraries on protein scaffolds. In S. Cabilly, Editor, Methods in Molecular Biology, vol. 87: Combinatorial Peptide Library Protocols, Humana Press, Totowa, N.J., USA, pp. 249-264.
31. Mattern, J., Koomaqi, R., Volm, M. 1996. Brit J Cancer 73:931-934.
32. McColley, S. A., et al. 2000. Am J Respir Crit Care Med 161:1877-1880.
33. Mendez, M. J., et al. 1997. Nat Genet 15:146-156.
34. Michels, S., Rosenfeld, P. J. 2004. Retinal Physician 1:16-22.
35. Michels, S., et al. 2005. Opthamology 112:1035-1047.
36. Murohara, T., et al. 1998. Circulation 97:99-107.
37. Presta, L. G., et al. 1997. Cancer Res 57:4593-4599.
38. Rosenfeld, P. J., Moshfeghi, A. A., Puliafito, C. A. 2005. Ophthalmic Surg Lasers Imaging 36:331-335.
39. Shields, R. L., Lai, J., Keck, R. 2002. J Biol Chem 277:26733-26740.
40. Shinkawa, T., et al. 2003. J Biol Chem 278:3466-3473.
41. Tamura, M., et al. 1998. Science 280:1614-1617.
42. Tomizuka, K., et al. 2000. Proc Natl Acad Sci USA 97:722-727.
43. Yancopoulos, G. D., et al. 2000. Nature (London) 407:242-248.

TABLE 1
Summary of XPA.10.064 and XPA.10.072 characteristics

| | XPA.10.064 | XPA.10.072 | Bevacizumab |
|---|---|---|---|
| HUVEC | 0.07 μg/ml | 0.68 μg/ml | 0.04 μg/ml |
| Proliferation | (0.66 in 2nd assay) | | |
| Potency (HPP): | | | |
| HEK293- | | | |
| hVEGF₁₆₅, IgG2 | | | |
| (IC50), | | | |
| HPP: sf21- | 0.41 μg/ml | >1 μg/ml | 0.06 μg/ml |
| hVEGF₁₆₅, scFvFc | | | |
| (IC50) | | | |
| HPP: sf21- | 0.15 μg/ml | 0.24 μg/ml | 0.05 μg/ml |
| hVEGF₁₆₅, IgG2 | (0.61 in 2nd assay) | | |
| (IC50) | | | |
| Binding to | 1.5 nM | 1.7 nM | 0.5 nM |
| hVEGF₁₆₅ (kD) | (IgG2) | (IgG2) | (IgG2) |
| Binding to | No binding | No binding | No binding |
| mVEGF₁₆₅ (kD) | | | |
| Human/murine | No | No | No |
| cross-reactivity | | | |
| Blocks binding of | Yes | Yes | Yes |
| hVEGF₁₆₅ to | (scFv-Fc) | (scFv-Fc) | |
| VEGF-R1 | | | |
| Blocks binding of | Yes | Yes | Yes |
| hVEGF₁₆₅ to | (scFv-Fc) | (scFv-Fc) | |
| VEGF-R2 | | | |
| Binds different | No | No | — |
| hVEGF₁₆₅ epitope | | | |
| than Bevacizumab | | | |
| Conformational | Linear | Linear | Linear |
| vs. linear | | | |
| epitope binding | | | |
| MPA (% αKLH | 20 | 26 | 38 |
| cntr; 2 × 5 mg/kg) | | | |

TABLE 2
Binding affinity of XPA.10.064
and XPA.10.072 IgG2 to hVEGF₁₆₅

| | ka (1/Ms) | kd (1/s) | Rmax (RU) | KD (M) | CHI2 |
|---|---|---|---|---|---|
| BM1 | 1.22E+05 | 7.38E−05 | 20 | 6.05E−10 | 0.251 |
| XPA.10.064 | 2.30E+05 | 2.86E−04 | 30 | 1.24E−09 | 0.379 |
| to Human | | | | | |
| XPA.10.072 | 1.68E+05 | 2.79E−04 | 29 | 1.66E−09 | 0.11 |
| to Human | | | | | |

TABLE 4
Antibody binding to hVEGF mutants:

| | CHO | wt 121 | M81A | G88S | Q89A | G92A | rHu-VEGF165 | rMu-VEGF165 |
|---|---|---|---|---|---|---|---|---|
| | 2 | 3 | 4 | 5 | 6 | 7 | 8 | 9 |
| PAB | 1 | 28 | 18.8 | 8.4 | 10.8 | 23.9 | 26.9 | 1 |
| | 0.09 | 2.5575 | 1.7145 | 0.769 | 0.9852 | 2.1765 | 2.4544 | 0.0939 |
| BM-1 | 0.9 | 39.4 | 13.1 | 0.9 | 5.3 | 35.6 | 36.7 | 0.9 |
| | 0.09 | 3.5953 | 1.1941 | 0.0828 | 0.4801 | 3.249 | 3.3501 | 0.0843 |
| XPA.10.064 | 0.9 | 29.1 | 9.5 | 0.9 | 3.5 | 22.8 | 23 | 0.7 |
| | 0.085 | 2.6504 | 0.8678 | 0.0778 | 0.3194 | 2.0792 | 2.0946 | 0.0667 |
| XPA.10.072 | 1.5 | 31.8 | 13.4 | 1 | 3.3 | 30.5 | 29.2 | 1.1 |
| | 0.142 | 2.902 | 1.2228 | 0.0951 | 0.2964 | 2.7809 | 2.6597 | 0.0982 |

(Note:
In this experiment, Abs were coated on the plate at 5 ug/ml; 100 ul of CHO cell supernatant for each mutant added, and Human and murine VEGF₁₆₅ used at 1 ug/ml. Background value is CHO. In each well upper number is ‘fold over background’, lower number is raw data.)

TABLE 5
Summary of epitope binding results

| | Binding to WT and Mutant Ligands | | | | | | |
|---|---|---|---|---|---|---|---|
| Antibody | wt 121 | M81A | G88S | Q89A | G92A | rHu-VEGF165 | rMu-VEGF165 |
| PAB | YES | YES | YES | YES | YES | YES | NO |
| BM-1 | YES | Reduced | NO | Weak | YES | YES | NO |
| XPA.10.064 | YES | Reduced | NO | Weak | YES | YES | NO |
| XPA.10.072 | YES | Reduced | NO | Weak | YES | YES | NO |

What is claimed is:

1. An isolated antibody or antigen-binding fragment thereof comprising,
1) a heavy chain variable region which comprises the following heavy chain CDR amino acid sequences: SEQ ID NO:6, SEQ ID NO:7 and SEQ ID NO:8, and/or
2) a light chain variable region which comprises the following light chain CDR amino acid sequences (a) SEQ ID NO: 9, SEQ ID NO: 10 and SEQ ID NO: 11, or (b) SEQ ID NO: 12, SEQ ID NO:13 and SEQ ID NO: 14.

2. The antibody of antigen-binding fragment thereof of claim 1 wherein the heavy chain variable region comprises the amino acid sequence set forth in SEQ ID NO:2.

3. The antibody or antigen-binding fragment thereof of claim 1 wherein the heavy chain variable region comprises an amino acid sequence set forth in SEQ ID NO:4.

4. The antibody or antigen-binding fragment thereof of claim 1 wherein the light chain variable region comprises the amino acid sequence set forth in SEQ ID NO: 3.

5. The antibody or antigen-binding fragment thereof of claim 1 wherein the light chain variable region comprises an amino acid sequence set forth in SEQ ID NO:5.

6. The antibody or antigen-binding fragment thereof as in any of claims 1-5 further comprising an IgG2 constant region.

7. The antibody or antigen-binding fragment thereof as in any of claims 1-5, wherein said antibody binds hVEGF₁₆₅ with a K_{D} of ≦2.0 nM.

8. The antibody or antigen-binding fragment thereof as in any of claims 1-5, wherein said antibody binds to an epitope on hVEGF₁₆₅ that overlaps with the epitope bound by Bevacizumab.

9. The antibody or antigen-binding fragment thereof as in any of claims 1-5, wherein said antibody blocks binding of hVEGF₁₆₅ to VEGF-R1 and VEGF-R2.

10. A method of inhibiting angiogenesis in a subject in need thereof comprising administering to said subject a therapeutically effective amount of the antibody or antigen-binding fragment thereof as in any of claims 1-5.

11. A method of treating a disease associated with aberrant angiogenesis in a subject in need thereof comprising administering to said subject a therapeutically effective amount of the antibody or antigen-binding fragment thereof as in any of claims 1-5.

12. A method of treating an inflammatory disease associated with VEGF signaling in a subject in need thereof comprising administering to said subject a therapeutically effective amount of the antibody or antigen-binding fragment thereof of claim 1.

13. The method of claim 12, wherein said disease is selected from the group consisting of rheumatoid arthritis, psoriasis, scleroderma, chronic obstructive pulmonary disease, and asthma.

14. A method of treating wet acute macular degeneration or diabetic retinopathy in a subject in need thereof comprising administering to said subject a therapeutically effective amount of the antibody or antigen-binding fragment thereof as in any of claims 1-5.

15. A method of treating a cancer associated with increased VEGF signaling in a subject in need thereof comprising administering to said subject a therapeutically effective amount of the antibody or antigen-binding fragment thereof of claim 1.

16. The method of claim 15, wherein said antibody or antigen-binding fragment thereof further comprises a conjugate.

17. The method of claim 16, wherein said conjugate is selected from the group consisting of a toxin, a cytokine, and a chemotherapeutic agent.

18. A kit comprising the antibody or antigen-binding fragment thereof as in any of claims 1-5.

19. An isolated polynucleotide encoding the amino acid sequence set forth in any of SEQ ID NOs:2, 3, 4, 5, 6, 7, 8, 9, 10, 11, 12, 13, or 14.

20. An isolated vector comprising a polynucleotide of claim 19.

21. An isolated host cell comprising a vector of claim 20.

22. A fully human antibody wherein the antibody comprises one or more characteristics selected from the group consisting of:
(1) binding to hVEGF₁₆₅ with a K_{D} no less than 10-fold lower than the K_{D} with which the antibody binds mVEGF_{165;}
(2) binding to hVEGF₁₆₅ with a K_{D} of ≦2.0 nM;
(3) binding to an epitope on hVEGF₁₆₅ that overlaps with the epitope bound by Bevacizumab;
(4) blocking binding of hVEGF₁₆₅ to a VEGF receptor; or
(5) inhibiting hVEGF₁₆₅-induced phosphorylation of a VEGF receptor.

23. The fully human antibody of claim 22 wherein the antibody binds to hVEGF₁₆₅ with a K_{D} of ≦2.0 nM.

24. The fully human antibody of claim 22 wherein the antibody binds to an epitope on hVEGF₁₆₅ that overlaps with the epitope bound by Bevacizumab.

25. The fully human antibody of claim 22 wherein the antibody blocks binding of hVEGF₁₆₅ to a VEGF receptor.

26. The fully human antibody of claim 25 wherein the VEGF receptor is VEGF-R1 or VEGF-R2.

27. The fully human antibody of claim 22 wherein the antibody inhibits hVEGF₁₆₅-induced phosphorylation of a VEGF receptor.

28. The fully human antibody of claim 22 wherein the antibody comprising a heavy chain variable region which comprises the following heavy chain CDR amino acid sequences: SEQ ID NO:6, SEQ ID NO:7 and SEQ ID NO:8.

29. The fully human antibody of claim 28 further comprises a light chain variable region which comprises the following light chain CDR amino acid sequences: SEQ ID NO:9, SEQ ID NO:10 and SEQ ID NO:11.

30. The fully human antibody of claim 29 wherein the heavy chain variable region comprises an amino acid sequence set forth in SEQ ID NO:2 and the light chain variable region comprises an amino acid sequence set forth in SEQ ID NO:3.

31. The fully human antibody of claim 28 further comprises a light chain variable region which comprises the following light chain CDR amino acid sequences: SEQ ID NO:12, SEQ ID NO:13 and SEQ ID NO:14.

32. The fully human antibody of claim 31 wherein the heavy chain variable region comprises an amino acid sequence set forth in SEQ ID NO:4 and the light chain variable region comprises an amino acid sequence set forth in SEQ ID NO:5.

33. The antibody or antigen-binding fragment thereof as in any of claims 1-5 which is a full antibody.

34. The antibody of claim 33 which is a monoclonal antibody.

35. The antibody of claim 33 which is a polyclonal antibody.

36. The antibody of claim 33 which is a recombinant antibody.

37. The antibody of claim 33 which is a bispecific antibody

38. The antibody of claim 33 which is a humanized antibody.

39. The antibody of claim 33 which is a chimeric antibody.

40. The antibody of claim 33 which is a fully human antibody.

41. An isolated human antibody comprising a light chain variable region comprising the amino acid sequence set forth in SEQ ID NO: 3 and a heavy chain variable region comprising the amino acid sequence set forth in SEQ ID NO: 2.

42. An isolated human antibody comprising a light chain variable region comprising the amino acid sequence set forth in SEQ ID NO: 5 and a heavy chain variable region comprising the amino acid sequence set forth in SEQ ID NO: 4.

43. A pharmaceutical composition comprising the antibody of claim 41 and a pharmaceutically acceptable carrier.

44. A pharmaceutical composition comprising the antibody of claim 42 and a pharmaceutically acceptable carrier.

45. A pharmaceutical composition comprising the antibody or antigen-binding fragment thereof as in any of claims 1-5 and a pharmaceutically acceptable carrier.

46. An isolated antibody or an antigen fragment thereof comprising at least one heavy chain complementarity determining region of XPA.10.064 or XPA.10.072 and at least one light chain complementarity determining region of XPA.10.064 or XPA.10.072.

47. The isolated antibody or the antigen fragment there of of claim 46 wherein the heavy chain complementarity determining region is selected from the group consisting of HCDR1, HCDR1, HCDR3, and combination thereof of XPA.10.064 or XPA.10.072.

48. The isolated antibody or the antigen fragment there of of claim 46 wherein the light chain complementarity determining region is selected from the group consisting of LCDR1, LCDR1, LCDR3, and combination thereof of XPA.10.064 or XPA.10.072.
